(19) United States
(12) Patent Application Publication
(10) Pub. No.: US 2021/0063972 A1
(43) Pub. Date: Mar. 4, 2021

(54) COLLABORATIVE HUMAN EDGE NODE DEVICES AND RELATED SYSTEMS AND METHODS

(71) Applicant: FACET LABS, LLC, Los Gatos, CA (US)

(72) Inventors: Lindsay SPARKS, Seattle, WA (US); Stuart OGAWA, Los Gatos, CA (US); Koichi NISHIMURA, San Jose, CA (US); Wilfred P. SO, Mississauga (CA)

(21) Appl. No.: 16/981,037

(22) PCT Filed: Mar. 15, 2019
(86) PCT No.: PCT/US19/22415

§ 371 Date: Sep. 15, 2020

Related U.S. Application Data
(60) Provisional application No. 62/643,413, filed on Mar. 15, 2018.

(51) Int. Cl. G05B 13/02; G16H 40/67; G06N 3/08; A61B 5/0476; A61B 3/113; A61B 5/04; A61B 5/145; A61B 5/11; A61B 5/00; A61F 2/72; A61F 4/00

(52) U.S. Cl. CPC G05B 13/027; G16H 40/67; G06N 3/08; A61B 5/0476; A61B 3/113; A61B 5/04001; A61F 2002/704; A61B 5/1114; A61B 5/4803; A61F 2/72; A61F 4/00; A61B 5/744; A61B 5/7475; A61B 5/145

(57) ABSTRACT

Biological human-computer interfaces are used to control devices, such a robotic limbs, using brain signals, nerve signals and muscle signals. These interfaces are sometimes called brain-computing interfaces. The learning process for a user to control devices using these interfaces is cumbersome. A computing platform is provided that includes a library of human model templates that are used to take biological inputs signals to generate outputs that control the device. Different templates are more appropriate for users of different attributes, such as demographic attributes. The computing platform collects data from multiple other users with these interfaces and devices in order to calibrate the human model templates. The calibrated human model templates are then published to the individual users, so that they can more accurately control their devices using their biological input signals.

### CROSS-REFERENCE TO RELATED APPLICATIONS

This application claims priority to U.S. Provisional Patent Application No. 62/643,413 filed on Mar. 15, 2018 and titled “Collaborative Human Edge Node Devices and Related Systems and Methods”, the entire contents of which are herein incorporated by reference.

### TECHNICAL FIELD

The following generally relates to improving the interaction between a living organism and a device by sensing or affecting, or both, the living organism's brain signals, nerve signals, muscle signals, other biological signals, etc. In a more particular embodiments, the living organism is a human.

### DESCRIPTION OF THE RELATED ART

Biological human-computer interfaces have been and continue to be developed to control devices, such as, but not limited to, robotic prosthetic limbs. These type of interfaces are also called brain-computer interfaces (BCI).

For example, a person with an amputated arm is equipped with a robotic prosthetic limb. The robotic prosthetic limb has a communication device that can receive control signals to control the actuators of the robotic prosthetic limb. A biological human-computer interface is able to acquire signals of the person's body (e.g. brain signals, nerve signals, muscle signals, etc.), which are also called intention signals; use a computing model to generate control signals from these intention signals, which is also called a decoding process; and transmit the control signals to the robotic prosthetic limb to cause the same to move.

Examples of biological human-computer interfaces include a neural-control interface (NCI), a mind-machine interface (MMI), a direct neural interface (DNI), a brain-machine interface (BMI). Sensors can be placed in the brain, on the brain, exterior to the skull, on a nerve, within a nerve, on the skin to detect nerve signals, within a muscle, on a muscle, and on the skin to detect muscle signals.

An example of sensors include an array of micro-electrodes implanted in the brain, which is in the field of electrocorticography. A specific example of such an array is a Utah Array. Another type of sensor includes electrodes pushed into the brain tissue.

Functional near-infrared spectroscopy (fNIRS) can also be used to obtain brain signals. fNIRs is the use of near-infrared spectroscopy (NIRS) for the purpose of functional neuroimaging. Using fNIR, brain activity is measured through hemodynamic responses associated with neuron behavior.

Electroencephalogram (EEG) headsets can also be used to read brain signals.

Another form of sensors include ultrasonic wireless neural dust motes that can be implanted in the central nervous system and the brain.

Another form of sensors include electromyographic (EMG) sensors that measure signals from the muscles.

It will be appreciated that the number and the types of sensors to detect biological signals of a human is growing and these currently-known and future-known biological sensors can be used in human-computing systems to help control devices. Biological signals herein refer to and include, for example, one or more of: brain signals, nerve signals, muscle signals, chemical signals, eye movements, micro-body movements, subvocalizations, etc., and combinations thereof. Other types of biological signals can be included.

Typically, these devices are custom made for a user in order to suit the user's specific characteristics and needs. The control systems for these devices depend on the types of biological signals being obtained from the person, depend on the device itself, and the attributes of the user. In other words, building and adapting human-computer interfaces and devices to a person is very difficult and time consuming. Furthermore, the process becomes more complex when trying to replicate this process for many different people (e.g. different ages, different genders, different body types, different biological signals, etc.).

It is herein recognized that a person's biological signals to control a device can change over time. As a result, a control system that uses a specific biological signal to control the device may be effective at first, but the same control system may no longer be effective if the person's biological signals fluctuate over time. This is could lead to a perceived lack of sensitivity of the human-computer interface, or a perceived loss of control. For example, a person with dementia or other neurological condition could have fluctuating biological signals. In the example of an aging person, the fine motor skills, muscle strength, range of motion, and cognitive abilities change from a toddler to a child to a teenager to an adult and to a senior. There may be other reasons and conditions that cause fluctuations in biological signals over time.

Further complexity is introduced when a user switches or upgrades their device. For example, a child uses a smaller robotic prosthetic limb at first and, when the child becomes a teenager, then uses a larger-sized robotic prosthetic limb. A new control system is required to control the larger-sized robotic prosthetic limb. In another example, a person with a robotic limb upgrades or switches a component within the robotic limb, which in turn changes the control characteristics of the robotic limb. Using the same control system to control the upgraded robotic limb could lead to inaccurate control.

From the person's perspective, trying to learn to control these devices using their mind or their thoughts becomes a difficult and time-consuming process. Furthermore, a person who has to re-learn how to use and control a device because of upgrades to hardware and software would find the re-learning process time-consuming, difficult and frustrating.

### BRIEF DESCRIPTION OF THE DRAWINGS

Embodiments will now be described by way of example only with reference to the appended drawings wherein:

FIG. 1 is a schematic diagram of an example collaborative human edge node system.

FIG. 2 is a schematic diagram of a given person's device interacting with modules on a computing platform, including a human model templates library for the given device.

FIG. 3 is an example of a human model template.

FIG. 4 is another example of a dynamic human model template that adaptively selects sub-templates in response to sensor data.

FIG. 5 is a schematic diagram of a multiple persons and their devices interacting with modules on a computing platform, including a human model templates library storing templates for different devices.

FIG. 6 is an example of computer executable instructions for collecting data about a person before a loss of a body part and, after the loss of the body part, using the collected data to build, train, calibrate, or a combination thereof, a human model template for controlling a prosthetic device.

FIG. 7 is an example of computer executable instructions for collecting data about a remaining complimentary limb subsequent to losing a limb, and using the collected data to build, train, calibrate, or a combination thereof, a human model template for controlling a prosthetic device.

FIG. 8 is an example showing the relationship between an inverse human model template and a forward human model template, which are used to control a given device.

FIG. 9 is an example of computer executable instructions for using a state estimation filter to update a human model template based on data collected from various users and their devices.

FIG. 10 is an example of computer executable instructions for using data collected from various users and their devices to train a neural network that classifies human movement, which in turn updates a human model template.

FIG. 11 is an example of computer executable instructions for using data collected from various users and their devices to train a neural network that classifies actions of a device, which in turn updates a human model template.

FIGS. 12a, 12b and 12c are examples of human model templates that each include a correction bot.

FIG. 13 is an example of computer executable instructions for using Internet data sources to determine a corrected output used to control a device.

FIG. 14 is an example of computer executable instructions for using Internet data sources and sensor data to determine a corrected output used to control a device.

FIG. 15 is an example of computer executable instructions for using oral data from a user to label an intended action in advance, thereby providing data to build, train, calibrate, or a combination thereof, a human model template.

FIG. 16 is an example of computer executable instructions for using the oral labelling process, as described in FIG. 15, to perform various actions to calibrate a human model template for a given user.

FIG. 17 is a schematic example of a brain-computer interface that control two robotic prosthetic limbs, and the control of each of the robotic prosthetic limbs takes into account the state of the robotic prosthetic limb.

FIG. 18 is a schematic example of a brain-computer interface that controls a drone, and a brain stimulating device augments the brains ability to control the drone.

FIG. 19 is a schematic example of a brain-computer interface that controls a muscle stimulating device, and a brain stimulating device augments the brains ability to control the muscle stimulating device.

FIG. 20 is a schematic example of a brain-computer interface that converts brain signals representing thought speech to audio data on an auditory device.

FIG. 21 is an example of computer executable computations for obtaining training data to train a human model template that converts brain signals to language data.

FIG. 22 is another example of computer executable computations for obtaining training data to train a human model template that converts brain signals to language data.

FIG. 23 is a schematic example of a brain-computer interface that converts brain signals representing thought speech to language data, and using these systems for non-verbal conversation between users.

FIG. 24 is a schematic example of a brain-computer interface that converts brain signals representing thought speech to language data, and using these systems to control a robot.

FIG. 25 is a schematic diagram of an example computing architecture for ingesting biological data and other sensor data, and providing big data computations and machine learning using a data enablement platform.

FIG. 26 is another schematic diagram, show another representation of the computing architecture in FIG. 25.

FIG. 27 is a schematic diagram showing an example computing architecture for a data enablement platform, which includes supporting parallelized collector bots.

### DETAILED DESCRIPTION

It will be appreciated that for simplicity and clarity of illustration, where considered appropriate, reference numerals may be repeated among the figures to indicate corresponding or analogous elements. In addition, numerous specific details are set forth in order to provide a thorough understanding of the example embodiments described herein. However, it will be understood by those of ordinary skill in the art that the example embodiments described herein may be practiced without these specific details. In other instances, well-known methods, procedures and components have not been described in detail so as not to obscure the example embodiments described herein. Also, the description is not to be considered as limiting the scope of the example embodiments described herein.

In an example embodiment, a human is working and thinking with an artificial intelligence (AI) enabled computer and is optionally connected to a robotic device(s) with the intent that the human drives a specific and directive set of device actions. The human can drive these action using their thoughts or intents, which are detected as brain signals, nerve signals, muscle signals, chemical signals, and other biological related signals.

For example, a person with a prosthetic body limb or an embedded electronic eye or ear could use their brain signals to drive these devices. Alternatively, or in combination, their brain signals are used to drive an external thing, such as a drone, a robot, a car, a virtual avatar in a virtual environment, etc.

It is herein recognized that a person is a controller of a device based on brain control, muscle control, nerve control, or other human means, or a combination thereof. The process for a person to learn an interaction with that device takes time. The brain needs to adapt, “rewire” itself, build memory when synapses are created, etc.

It is additionally herein recognized that this problem is amplified by a larger number of different people trying to learn to control the same device (e.g. same model of robotic prosthetic arm), since different people learn differently. The same model of the robotic prosthetic arm cannot be generalized to everyone's learning style. For example, different demographic attributes, size attributes, muscle attributes, central nervous system attributes, etc. of people will affect how biological signals are generated and how a robotic prosthetic arm should move.

The same control system of a given device should not be used for different people using the same device. For example, a brain-controlled robotic arm for a 50 year old man should not operate in the same way as a brain-controlled robotic arm for a 7 year old girl. The movements of an arm of a 50 year old man would be different from the movements of an arm of a 7 year old girl.

This problem is also amplified by the very large variety of devices that people could control.

Devices, systems and methods are provided for accelerate the learning process between human control of any given device using collaborative data from other human-computer interfaces. In an example embodiment, machine learning, also including AI, could be used to obtain aggregated data to provide and select human model templates for controlling devices, and update these templates as more data becomes available. These human model templates are used to control a device based on data received by a human computer interface. In other words, the devices controlled by human computer interfaces accurately match a given user (and their attributes), and the given user is able to more quickly and accurately control these devices with their thoughts or intentions.

In another example embodiment, the embedded BCI that controls the limbs, IoT devices, or other type of device, is used to stimulate new neurons, synapses, and axions to accelerate the brain in re-wiring its own neural connections. For example, in the context of playing music, playing the A major scale has three sharps. Assume the person has certain brain damage that prevents the person from playing the three raised sharps. An embedded device (e.g. BCI) sends or transmits broad signals to parts of the brain to stimulate new circuits to play the 3 raised sharps.

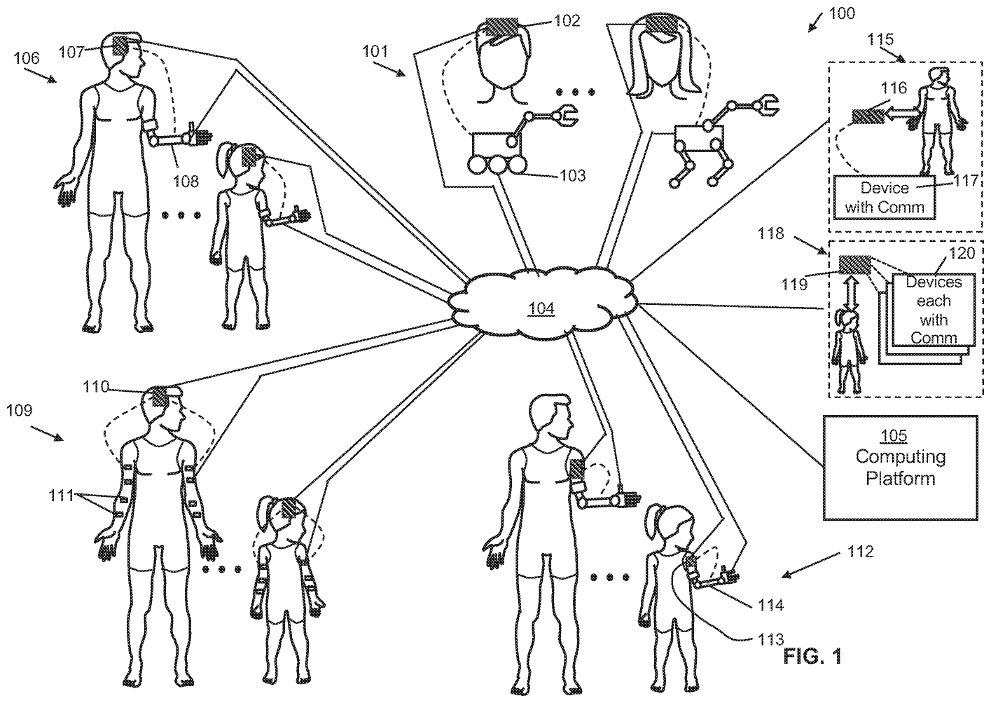

Turning to FIG. 1, a collaborative human edge node system 100 is shown in which data from various human edge node devices are be shared with each other, either in a peer-to-peer manner over a data network 104, or via a computing platform 105, or both. The data from each of the human edge node devices are processed to improve human model templates that are used to control certain devices.

In an example embodiment, human edge node devices include human-computer interface devices that obtain biological signals. The human edge node devices also include devices that are controlled in response to the obtained biological signals.

An example group of users 101 each have a BCI 102 to control a drone 103. The drone 103, for example, is remotely controlled (e.g. such as in a mine, a remote building, another country, etc.) using brain signals sensed by the BCI 102. This control from the BCI 102 to the drone 103 is shown by the dotted line between these two components, which forms a direct communication or an indirect communication via the network 104. The BCI 102 has one or more sensors, one or more onboard processors, and one or more communication devices. The BCI 102 also has, for example, its own devices to affect the brain or the nervous system of the person. The drone 103 has sensors, one or more processors, one or more communication devices, and one or more actuators. Data from the BCI 102 and the drone 103 are transmitted to the computing platform 105 for data aggregation and processing. The people in the group of users 101 could all be controlling similar types of drones, but the individual persons could be very different from each other.

Another example group of users 106 each have a BCI 107 to control a robotic prosthetic limb 108 on their body. This control from the BCI 107 to the robotic prosthetic limb 108 is shown by the dotted line between these two components, which forms a direct communication or an indirect communication via the network 104. The BCI 107 has similar components to the BCI 102, although the form of these components can be different. The robotic prosthetic limb 108 has one or more sensors, one or more processors, one or more communication devices and one or more actuators. Data from the BCI 107 and the robotic prosthetic limb 108 are transmitted to the computing platform 105 for data aggregation and processing. The people in the group of users 106 could all be controlling similar types of robotic prosthetic limbs, but the individual persons could be very different from each other.

Another example group of users 109 each have a BCI 110 to control one more muscle stimulating devices 111 on their body. This control from the BCI 110 to the robotic muscle stimulating devices 111 is shown by the dotted line between these two components, which forms a direct communication or an indirect communication via the network 104. The BCI 110 has similar components to the BCI 102, although the form of these components can be different. The muscle stimulating devices have one or more stimulating components, one or more processors, and one or more communication devices. Preferably, there are one or more sensors amongst the muscle stimulating devices to obtain feedback about the muscle. Data from the BCI 110 and the muscle stimulating devices 111 are transmitted to the computing platform 105 for data aggregation and processing. The people in the group of users 109 could all be controlling similar types of muscle stimulating devices, but the individual persons could be very different from each other.

Another example group of users 112 each have a muscle or nerve computer interface 113 located at the upper arm to control a robotic lower limb 114. The interface 113 has sensors to measure nerve signals or muscle signals, or both, which in turn are processed and used to control the actuators of the robotic lower limb 114. This control is shown by the dotted line between these two components, which could be data or commands transmitted using a direct communication or an indirect communication via the network 104. The interface 113 has one or more sensors, one or more onboard processors, and one or more communication devices. The interface 113 also has, for example, its own devices to affect the muscle or the nerves of the person, which provides feedback to the person. The prosthetic limb 114 has one or more stimulating components, one or more processors, one or more actuators, and one or more sensors. Data from the interface 113 and the robotic lower limb 114 are transmitted to the computing platform 105 for data aggregation and processing. The people in the group of users 112 could all be controlling similar types of muscle stimulating devices, but the individual persons could be very different from each other.

In an example embodiment, the interface 113 is a standard “bus” or fitting so that different devices (e.g. different robotic prosthetic devices) can be removably attached to the interface.

In a more general example 115, a human-computer interface 116 is in communication with a device 117 that also has a data communication system. The human-computer interface 116 interfaces with some part of the person. The human-computer interface 116, for example, does not touch the person and remotely interfaces with the person. In another example, the human-computer interface 116 touches the person. In another example, the human computer interface 116 is embedded in the person. In an example embodiment, the device 117 and the human-computer interface 116 have a bi-directional communication link. The person, via the human-computer interface 116, can send data signals to the device 117, and the device 117 can send data signals to the human-computer interface 116. One or both of the human-computer interface 116 and the device 117 are in communication with the computing platform 105 via the network 104.

In another example 118, a human-computer interface 119 is in communication with multiple devices 120 that each have a data communication system. The human-computer interface 119, for example, does not touch the person and remotely interfaces with the person. In another example, the human-computer interface 119 touches the person. In another example, the human computer interface 119 is embedded in the person. In an example embodiment, the multiple devices 120 and the human-computer interface 119 have a bi-directional communication link. The person, via the human-computer interface 119, can send data signals to the devices 120, and the devices 120 can send data signals to the human-computer interface 119. The human-computer interface 119 or the devices 120, or both, are in communication with the computing platform 105 via the network 104. In this configuration, a person is able to use the human-computer interface 119 to control multiple devices 120 at the same time, or one at a time in sequence, or both. In an example embodiment, the multiple devices 120 form a swarm of devices that co-operate together to achieve a task. In an example embodiment, the multiple devices are the same type of device. In another example embodiment, the multiple devices are a collection of different types of devices.

For example, the devices 117 and 120 are Internet of Things (IoT) devices. In another example aspect, the devices 117 and 120 are respectively controllable by the interfaces 116 and 119. In another example aspect, both the devices 117, 120 and the interfaces 116, 119 have upgradable software, including and not limited to machine learning capabilities.

Human-computer interfaces are used to interface with a person. Non-limiting examples of human-computer interfaces include brain control interfaces; interfaces that measure muscle signals; interfaces that measure chemical signals; interfaces that measure chemical signals; interfaces that measure micro-body movements; interfaces that measure subvocalizations; interfaces that measure movement of the larynx or tongue or jaw, or a combination thereof; interfaces that measure nerve signals; interfaces that measure body part movement; interfaces that measure body attributes (e.g. heart rate, blood pressure, temperature, etc.); interfaces that measure eye movement; and interfaces that measure internal body part movement. It will be appreciated that there are many types of currently known and future-known human-computer interfaces that are applicable to the principles described herein.

In an example embodiment, a human-computer interface tracks eye movement. The human-computer interface, for example, obtains digital images of the eye(s) (e.g. via a camera) to track the movement of the eye(s). The digital images could include images captured in the visible light spectrum or images captured in the infrared or near infrared light spectrum, or a combination thereof. Other types of eye-tracking sensors include sensors attached to an eye. For example, a contact lens with an embedded sensor measures the activity of the eye. Another type of eye-tracking sensor measures electric potentials around the eyes. Other types of sensors of tracking eye movement are applicable to the principles described herein.

In an example embodiment, a human-computer interface tracks movement of internal body parts (e.g. lungs, bowels, heart, blood vessels, and other internal organs).

In general, a human-computer interface, some examples which have been described above, can be used to control various physical devices and virtual devices or bots. Non-limiting examples of physical devices include IoT devices, robotic devices, drones, exoskeleton devices, robotic prosthetic limbs, computing devices connected to display devices, computing devices with an audio speaker, media projectors, vehicles, communication devices, medical devices, devices that affect a body (e.g. muscle stimulating device; brain stimulating device; nerve stimulating device; chemical stimulating device; device that releases one or more drugs; device that stimulates at least one of new neurons, new synapses, and new axions to stimulate new neural circuits, etc.), tablets, mobile devices, desktop computers, laptops, etc. Virtual devices include virtual avatars of people, virtual avatars of things, and graphical user interface control elements (e.g. a button, scrolling a screen, swiping on a screen, enlarging a screen, typing on a screen, moving a cursor on a screen, etc.).

In the above examples, each of the human-computing interfaces have their own onboard processor and onboard communication device and each of the devices have their own onboard processor and onboard communication device. In an example embodiment, each of these interfaces and each of these devices communicate directly with the network 104. In another example, in a system or a pairing of an interface and a device (or multiple devices) controlled by the interface, only one of these components is designated to communicate directly with the network 104. In an example embodiment, the onboard processors on the respective devices or interfaces, or both, are considered system on chips (SOCs). Non-limiting examples of these onboard processors include ASICs, FPGAs, Tensor Processing Units (TPUs), Graphics Processing Units (GPUs), neuromorphic chips, quantum processors, and other types of currently known and future known processors. In an example embodiment, quantum processors are used to secure communication of data between interfaces and devices, as the data can be very sensitive to the safety and privacy of a person. It will be appreciated that the communication devices provide for wireless communication. In other examples, the communication devices provide wired communication, or both wired and wireless communication.

In the illustrated examples, a given person uses one human-computing interface to control a device. However, in other examples, a given person can use multiple human-computing interfaces to control a given device. For example, a blend or fusion of different biological related data (e.g. combinations of any of eye movement tracking, brain signals, muscle signals, heart rate, limb movement, micro-body movement, subvocalizations, etc.) is sensed from one or more human-computing interfaces, and this fusion of data is used to control a device.

It will be appreciated that there are other currently known interfaces, other currently known devices, future-known interfaces and future-known devices. Therefore, the many other combinations of interfaces and devices are applicable to collaborative human edge node system 100.

Based on the above, it will also be appreciated that there could be thousands or millions of human edge node devices that form the system 100 and that exchange data with the computing platform 105.

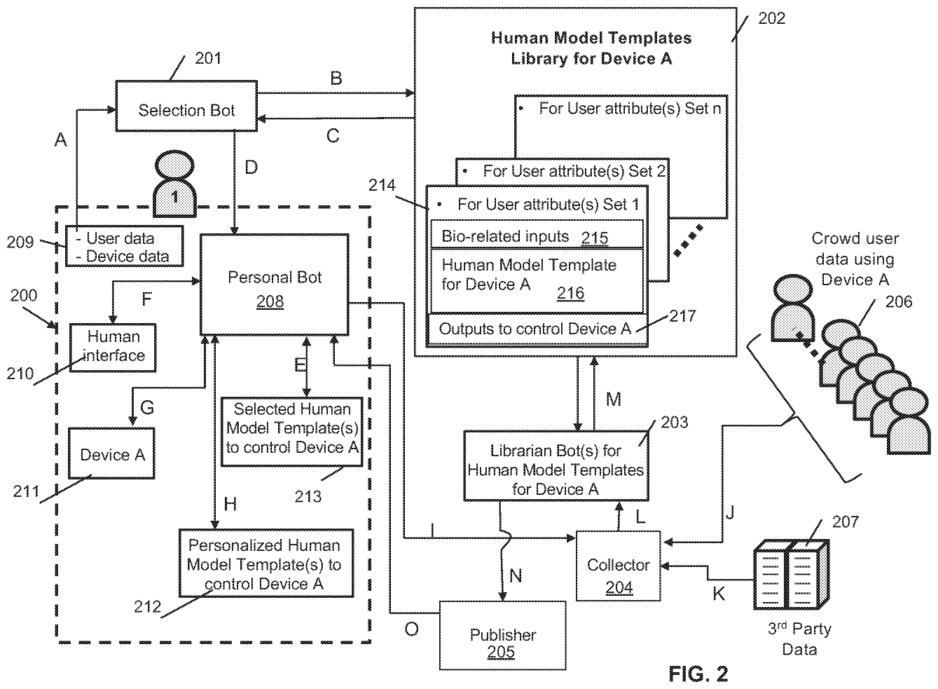

Turning to FIG. 2, an example embodiment of devices and executable modules are shown, including the flow of data between these components. The example system in FIG. 2 is for edge node device 200 of User 1 selecting an appropriate human model template that will be used to control Device A 211 of User 1.

In particular, the hardware and the software components within the dotted line 200 represent the components of the edge node of User 1. The group of other human edge node device 206 that user a same type of Device A, are also shown in FIG. 2. The remaining components in FIG. 2 are part of the computer platform 105.

As used herein, a human model template is a set of computations that convert bio-related inputs (e.g. brain signals, nerve signals, muscle signals, chemical signals, eye movements, micro-body movements, subvocalizations, etc.) to generate outputs that control a device in the manner intended by the person (e.g. User 1) that generated those bio-related inputs. In an example embodiment, the human model template could also receive other types of inputs from sensors embedded on the device or sensors positioned externally, in order to generate the outputs to control the device. The set of computations is called a model, as it takes into account, amongst other things, the characteristics of the device being controlled. These characteristics of the device are also representable as a plant model. The set of computations also models the control system, and the interaction between the control system and the plant model of the device. The control system is herein made to be customized to the attributes of a person. Other embodiments of a system model include a control system interacting with one or multiple devices, and receiving feedback in relation to these devices. The feedback, for example, is from sensors that measure the effects caused by the action of these devices. The feedback, in another example, is from sensors that measure the device's actions or device's characteristics.

In an example aspect, data science algorithms reside here in the template. In another example, compound algorithms reside here in the template. In another example, machine learning and neural networks reside here in the templates. These computations are, for example, wirelessly flashed to the SOC in the device(s) or interface(s) of the human edge node, where the SOC runs the human model template.

For devices that move, the plant model can include kinematic model or a dynamic model, or both. For devices that cause electrical actions, light actions, chemical actions, then a different plant model is used. Machine learning is, for example, used to determine the real time, unknown inbound biological related signal (e.g. electrical or light or chemical or any combinations of the aforementioned) and consequently autonomously apply the right kinematic models or dynamic models, or combinations thereof.

It is herein recognized, that even for the same device (e.g. a robotic limb) being used by different people, the human model template would vary between different people as they have different personal attributes, also herein referred to as user attributes. In other words, the set of computations in the human model template take into account these different personal attributes.

Non-limiting examples of personal attributes include: age, gender or sex, height, weight, dimensions of a given body part, muscle tone, reflexes, common activities of the person, lifestyle information, ethnicity, heart rate, DNA data, neural pathway properties, hormonal properties, genetic properties, familial relationships, social relationships, health records, etc.

Familial relationships and social relationships or groups could be used, for example, on the basis that a person would share attributes with other people in the same family or the same social group, or both. For example, peoples bodily movements and people's thinking patterns have higher chances of being similar if they are in the same family or social group, or both. It will be appreciated that familial relationships and social relationships can be automatically determined by querying social network data, health records, ancestry databases, etc.

In FIG. 2, a human model templates library 202 for a given device (e.g. Device A) is provided that includes multiple human model templates for Device A. Each of these templates vary based on one or more personal attributes. For example, one of the templates 214 is customized or configured for a given user having user attribute(s) Set 1. The template 214 includes one or more parameters defining the type of bio-related input(s) 215 to be received, the actual human model template to control Device A 216, and one or more parameters defining the type output(s) 217 to be outputted to control Device A.

Depending on the human-computer interface (e.g. sensors, initial processing, etc.), the bio-related inputs being provided may be different. Accordingly, there are different human model templates to account for different human-computer interfaces and different user attributes.

The components and the overall process is described in a below example scenario.

User 1 is newly equipped with a human-computer interface 210, which detects User 1's intentions. User 1 is also equipped with a device (e.g. Device A) 211 that is controllable by the human-computer interface 210. Data and software modules belonging or ascribed to User 1 are stored in the memory in one or more of the following: User 1's human-computer interface 210; User 1's device A 211; and an external device (e.g. User 1's computing device, one or more external human edge nodes, the computing platform 105, etc.).

User 1's human edge node 200 includes the hardware, software and data associated with one or more of the human-computer interface 210, the corresponding Device A 211, the user data and device data 209, the personal bot 208, the selected human model template(s) to control Device A 213, and the personalized human model template(s) to control Device 212. In a further example embodiment, the human edge node includes the hardware, software and data associated with all the components 210, 211, 209, 208, 213, 212.

Initially, User 1's human edge node 200 has a default human model template that provides some basic control of Device A 211. Alternatively, the human edge node 200 is not provisioned with any human model template. To select an appropriate human model template for User 1, at Operation A, a selection bot 201 obtains user data and device data 209.

The selection bot 201, for example, is part of the computing platform 105 and has intelligent computations that selects the appropriate human model template from the library 202. In an example embodiment, the human-computer interface 210 or Device A automatically triggers communication with the selection bot 201, or User 1's interaction with the human-computer interface 210 or Device A 211 (or both) trigger communication with the selection bot 201.

After the selection bot 201 obtains the user data and the device data 209, the selection bot runs a query of the library 202 to select the most appropriate human model template for User 1 (i.e. Operation B). The selected human model template is copied from the library 202 (i.e. Operation C) and is provisioned into User 1's node 200, particularly to their personal bot 208 (i.e. Operation D).

In an example embodiment, the selection bot 201 selects multiple templates for User 1 and sends the multiple templates to User 1's node 200. For example, multiple templates may be appropriate for User 1. In another example, multiple templates are used to control Device A 211 based on the human-computer interface 210.

The personal bot 208 stores the selected human model template 213 (i.e. Operation E). The personal bot also customizes the selected human model template 213 for the User 1 (e.g. providing security features in the template, provisioning a log of the use of the template, provisioning version histories, etc.). The personal bot also provisions the human model template 213 so that it is integrated with the human interface 210 or Device A 211, or both.

Over time, the personal bot receives data about the human-computer interface 210 and Device A using the initially selected human model template 213 (i.e. Operations F and G). The personal bot uses this data as feedback to further personalize the computations and parameters of the selected human model template, thereby generating further versions and iterations of a more personalized human model template 212 to control Device A (i.e. Operation H). In this way, the personal bot ensures that User 1's control of Device A accurately corresponds to User 1's intentions as measured by the human-computer interface 210.

The feedback data obtained by the personal bot 208 in relation to the human interface 210 and Device A 211 is transmitted from the personal bot to a collector module 204 (i.e. Operation I). The feedback data includes raw data, derivatives of the raw data, the changes made by the personal bot to generate more personalized version of the human model template, or a combination thereof. The feedback data is also tagged with the user attributes and device attributes 209, which could be subject to change over time.

The collector module 204, also herein referred to as a collector, also collects data from other human edge nodes 206, in this example, using Device A (i.e. Operation J). These other nodes 206 are part of the collaborative human edge node system.

The collector 204 also collects data from third-party data sources 207 (i.e. Operation K). Non-limiting examples of third-party data sources include databases from other human device systems, device systems, drone systems, etc., which provide human data (e.g. biological data, personal attribute data, etc.) and device data itself (e.g. genres of devices, device attribute data, device performance data, device recall data, device quality data, device functionality data, etc.). These third-party data sources include publicly available data sources and privately available data sources. Examples of data sources could be Internet data of recordings of human movement of limbs, people talking, robotic movement of prosthetics, robotic movement of drones and other autonomous or remote-controlled devices, etc. These recordings could be in the form of video data, audio data, numerical data, text data, social network data, machine data, health data, biological data, sensor data, etc. Another example of a third-party data source are other software programs that are loaded and programmatically run to perform one or more actions (e.g. skills of a person, motions of a person, device actions, language actions, expression actions, musical actions, etc.). An action could be a singular action or a combination of different actions. In an example embodiment, the collector 204 includes a collector bot itself, or a system of collector bots.

The collector 204 ingests and pre-processes this data for storage and for access by the one or more librarian bots 203.

The librarian bot 203 obtains data pertinent to the control of Device A from the collector 204 (i.e. operation L) and uses this information to at least one of: modify human model templates, train human model templates, delete human model templates, and build new human model templates. In an example aspect, the librarian bot 203 uses machine intelligence to update the one or more human model templates based on the information obtained by the collector 204 (i.e. Operation M). This updating process could be continuous or occur at timed intervals. The updated human model templates make the control process of Device A more accurately reflect the true intentions of User 1.

The librarian bot 203 provides the one or more updated human model templates to a publisher module 205 (i.e. Operation N), also herein called a publisher, and the publisher 205 transmits the one or more updated human model templates to the relevant human edge nodes. In particular, the publisher 205 has computing processes that determine which particular updated human model templates should be transmitted to which particular human edge nodes. In the example of FIG. 2, the publisher 205 transmits a certain updated human model template to the human edge node 200 of User 1 (i.e. Operation O).

In response, the personal bot 208 receives this updated human model template and incorporates this updated human model template into control the human interface 210 or Device A 211, or both. The incorporation process includes, for example, adapting any previous personalizations that are specific to User 1. This closes the feedback loop from the collaborative human edge network to User 1.

It will be appreciated that Device A could be: a specific device (e.g. a certain device make); a specific system of integrated devices (e.g. a certain system make); or a genre of device (e.g. not limited to one manufacturer or model).

In an example where Device A represents a genre or class of device, then the user or the device itself provides the personal bot 208 or the selection bot 201, or both with Device A's input parameters, output parameters, and characteristics (e.g. what it does, degrees of freedom, number of actuators, power requirements, etc.). In this way, the personal bot can more accurately personalize the selected human model template, or the selection bot can more accurately select an appropriate human model template, or both.

It is recognized that as more companies and individuals are building and customizing their own devices that can be controlled by human-computer interfaces, it is desirable to have human model templates that are appropriate to a genre or general class of devices. These genre or class based templates can be adapted to these newer devices or niche devices, which in turn help these devices to be more readily used by people.

It will be appreciated that the term “bot” is known in computing machinery and intelligence to mean a software robot or a software agent. The bots described herein have artificial intelligence. For example, the librarian bot and the personal bot have artificial intelligence.

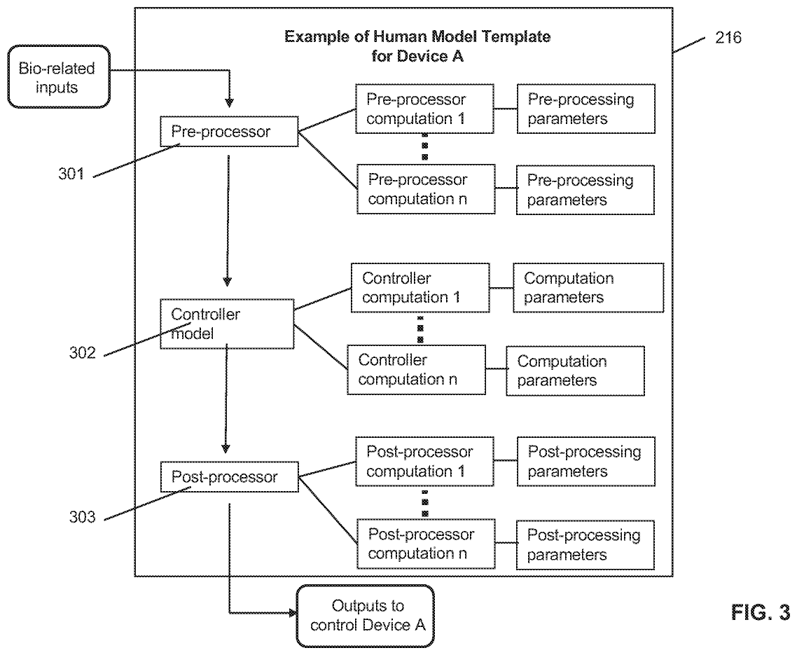

Turning to FIG. 3, an example of a human model template 216 for Device A is shown in more detail. The template 216 receives bio-related inputs according to a certain format and type, and generates outputs to control Device A according a certain format and type. The librarian bot 203 is able to modify the required format and type of inputs, and modify format and type of outputs being generated.

The template 216 includes a pre-processor 301, a controller model 302 and a post-processor 303. The inputs are pre-processed by the pre-processor 301 to generate pre-processed data. For example, the pre-processor performs computations for intention decoding based on the bio-related signals. The pre-processed data is inputted into the controller model 302 to generate intermediary control data for Device A. The intermediary control data is inputted into the post-processor 303 to generate outputs commands or signals that control Device A.

In another example embodiment, the functionality of the pre-processor and the post-processor are incorporated into the controller model.

The pre-processor 301 is associated with an available set of various pre-processor computations suitable for the controller model 302 and Device A. Each one of these available pre-processor computations is associated with one or more pre-processing parameters. In an example embodiment, the librarian bot 203 modifies the pre-processor 301 by selecting one or more of the pre-processor computations from the available set of pre-processor computations, and by adjusting the pre-processing parameters associated with the selected one more pre-processor computations. In this way, variants of the pre-processor can be customized for people of certain personal attributes.

The controller model 302 is associated with an available set of various controller computations that are each suitable for controlling Device A. Each one of these available controller computations is associated with one or more computation parameters. The librarian bot 203 modifies the controller model 302 by selecting one or more of the controller computations from the available set of controller computations, and by adjusting the corresponding computation parameters associated with the selected one more controller computations. In this way, variants of the controller model can be customized for people of certain personal attributes.

The post-processor 303 is associated with an available set of various post-processor computations that are each suitable for the controller model and Device A. Each one of these available post-processor computations is associated with one or more post-processing parameters. The librarian bot 203 modifies the post-processor 303 by selecting one or more of the post-processor computations from the available set of post-processor computations, and by adjusting the corresponding post-processing parameters associated with the selected one more post-processor computations. In this way, variants of the post-processor can be customized for people of certain personal attributes.

In an example embodiment, 301, 302, and 303 individually or in any combination reside locally on human edge node 200 as a system on chip (SOC). This results in faster responsiveness, including updating the computations or the parameters, or both. For example, the above-noted updates are made by wirelessly flashing the SOC.

The librarian bot 203 preferably can select or adjust computations, or both, and further adjust the parameters within those computations.

Alternatively, the computations in the pre-processor, the controller model and the post-processor are fixed, and the librarian bot 203 only modifies the parameters within these computations.

Therefore, the librarian bot is able to make various versions of the human model templates for Device A, and also iteratively update the human model templates for Device A as more information is collected. The personal bot 208 can also make these adjustments to a human model template at the given human edge node 200 in order to make the human model template personalized for User 1 at any present moment.

In this way, the templates stored in the library 202 accurately reflect models for various user attribute groups, so that a person classified within a certain user attribute group can have their human edge node device obtain a human template model that accurately processes their intentions to control Device A. In this way, the learning and training effort for that person to control Device A becomes faster and easier. Furthermore, as templates are updated over time, the person's control over Device A becomes more accurate and efficient. In another aspect, templates are updated over time by the personal bot 208 and the librarian bot 203 to accommodate the person's change (and people's change) in biological signaling, or the person's change (and people's change) in physical attributes, or both. For example, people's neural pathways change over time, their hormonal composition change over time, their neural conductivity changes over time, their brain activation changes over time, and their muscle reflexes change over time. Also, people can grow bigger or smaller in size over time, can increase or decrease in weight over time, can grow more or less muscular over time, amongst other physical changes. In this way, even as a person changes over time, the human model templates are updated to maintain accurate control of Device A that reflect the person's intentions.

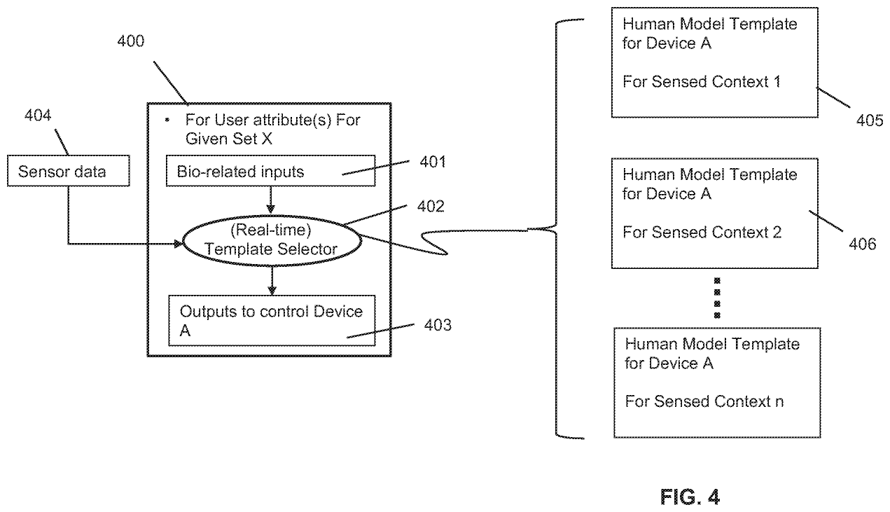

FIG. 4 shows an example of a dynamic human model template 400 to control Device A suitable for a user with a given set of attributes. A template selector bot 402 receives bio-related inputs 401 and other sensor data 404, and dynamically selects a human model template from a set of available templates (e.g. templates 405, 406) that are appropriate to the sensed context. The selected template is used to process the inputs 401 and generate outputs 403 to control Device A.

The purpose of the template selector bot 402 is to sense data about the user and the external environment of the user or the device itself (e.g. whereby the device is not attached to the user) and to determine the context of a user intention. Then, the template selector, preferably in real-time, selects the human model template for the given user (e.g. User 1) that matches the sensed context. As some examples of context, the template selector bot 402 uses the sensor data 404 to classify in real-time that the present context of the user is that the user is in a: relaxing mode; a sport mode; an excited social mode; an action-based work mode; a delicate and precise mode; a gentle mode; and an aggressive mode, amongst others. In this way, the control mode of a device (e.g. a robotic limb, a remotely controlled robot, a drone, etc.) matches the user's intention as appropriate to the determined context. For example, a person with a robotic prosthetic arm device has intentions to control this device in one way when playing tennis (e.g. a first template), and in a very different way when caressing a kitten (e.g. a second template), and further in a very different way when playing plano (e.g. a third template).

In an example embodiment, the sensor data 404 could come from sensors that are positioned on the device itself. For example, a device has sensors positioned on it to sense context, or the environment that it is in. Examples of such sensors include camera sensors, audio sensors, RADAR sensors, LiDAR sensors, tactile sensors, temperature sensors, etc. The device, for example, also has one or more processors to locally process the sensed data.

For example, video data or still image data (also called visual data) from one or more cameras mounted on a robotic limb are processed using image recognition computations. Visual data of a tennis racket, a tennis ball, and a tennis court indicate that the user is playing a game of tennis, and the template selector 402 selects a human model template that provides quicker reflex and more powerful movements to match the sport mode context. The template selector could further make the sport mode determination by detecting bio-related inputs in addition to the visual data, such as increased body temperature, increased blood pressure, and increased heart rate. By comparison, visual data of a kitten and optionally combined with decreased heart rate data indicate that the user is caressing a kitten. In response, the template selector 402 selects a human model template that provides slower reflex and more less forceful movements to match the gentle mode context.

It will be appreciated template selector bot can have machine learning computations to use the sensor data 404 to classify the present context. For example, the machine learning computations include, but are not limited to, neural networks, fuzzy logic, Bayesian classifiers, and K means.

In an example aspect, the computations and the parameters used by the template selector are updated over time in the library 202. In addition or in alternative, the computations and the parameters used by the template selector bot are updated on the human edge node 200 to become personalized to User 1.

Similarly, the context-specific human model templates 405, 406 can be updated over time in the main library 202, or updated and personalized for a given user on the human edge node 200, or both.

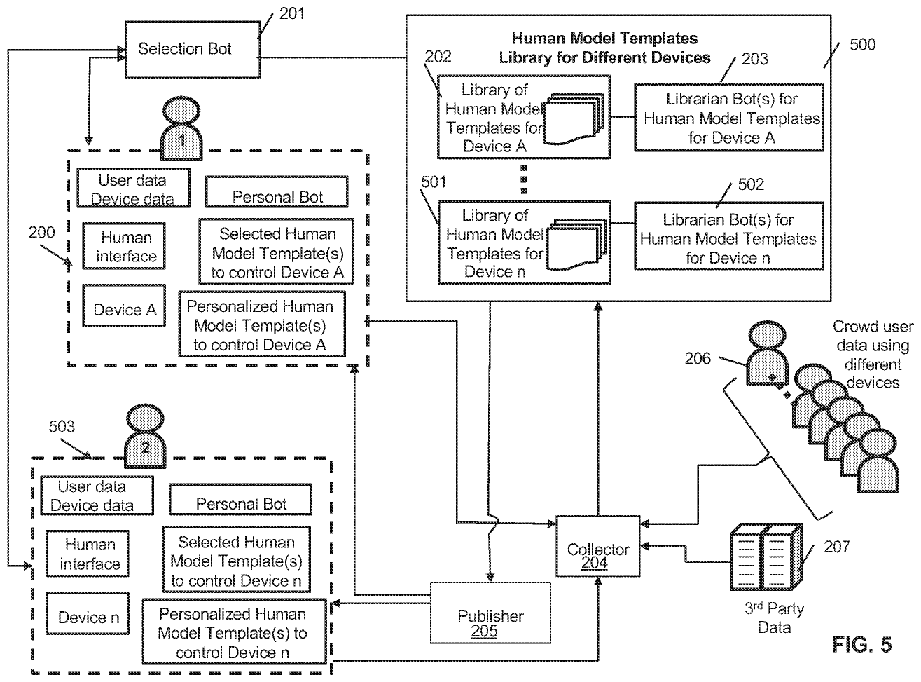

FIG. 5 shows a schematic similar to FIG. 2, but scaled larger for multiple types of devices and many different users. The system in FIG. 5 has the same features and functions. For example, User 2's human edge node 503 could have a different human-computing interface and a different device (e.g. Device n). The selection bot 201 also selects an appropriate human model template for User 2. A larger library 500 includes multiple smaller libraries for different devices. For example, the library for Device A 202 and the corresponding librarian bot 203 are part of the larger library 500. Another library of human model templates of Device n 501 and the corresponding librarian bot 502 are also part of the larger library 500. Furthermore, the crowd user data 206 comes across the overall collaborative network of human edge nodes that are using a variety of different devices and different human-computing interfaces.

The following is a more detailed discussion of the selection bot 201.

The selection bot 201 can use one or more types of computations or algorithms to select an appropriate human model template based on the provided data 209 (e.g. user data, device data, etc.). These computations are based on matching a given human edge node to one or more human model templates. Various types of current known and future known matching algorithms can be used to make a selection.

In an example implementation, the human model templates are tagged with predefined user attributes and predefined device attributes. The selection bot 201 identifies the human model that is tagged with the attributes that match the provided data 209.

In another example implementation, the selection bot 201 utilizes bipartite graphs to compute bipartite matching computations. For example, users represent one set of nodes and the templates represent another set of nodes in a bipartite graph. In an example embodiment, unweighted bipartite graphs are used to perform the matching. In another example, weighted bipartite graphs are used to perform the matching.

In another example implementation, the selection bot 201 utilizes fuzzy matching algorithms.

In another example implementation, the selection bot 201 uses look-alike algorithms to match a user with a human model template. For example, the selection bot 201 has processed the existing data to identify that many users having personal attributes and device attributes of the set [X] use the human model template Y. Therefore, the selection bot determines that a potential user that also has the attributes [X] should use the human model template Y. It will be appreciated that different attributes can be weighted differently.

In another example implementation, the selection bot 201 uses a neural network to predict (or output) which human model template will best match a user and their device. The neural network is trainable based on existing data of users and their templates.

In another example implementation, the selection bot 201 computes mutual information between a given attribute (or given attributes) of a user and a given attribute (or given attributes) of a human model template. The mutual information value measures the mutual dependence between two seemingly random variables. The higher the mutual information value, the higher correlated are these variables, which can be used to determine that a given user and a given human model template are a matching pair.

In another example implementation, the selection bot 201 computes one or more Pearson Correlation Coefficients (PCC) between a given attribute of a user and a given attribute of a human model template. The one or more PCCs are used by the selection bot 201 to make a selection of a human model template.

Other currently known and future-known matching algorithms can be used. It will also be appreciated that multiple matching algorithms can be combined together in order for the selection bot 201 to make a selection.

In an example embodiment, the human model templates library 500 includes teaching model templates specific to a cohort of users and their devices. For example, the teaching model templates identify areas where the user is lacking effective control over their device via the human-computer interface and provide a series of exercises for the user to improve their control abilities over the device. The exercises, also called lesson plans, help give active feedback to the user, via their human edge node, as they provide biological related inputs (e.g. brain signals, muscle signals, micro-body movements, nerve signals, sub-vocalizations, chemical signals, etc.) to try and control their device.

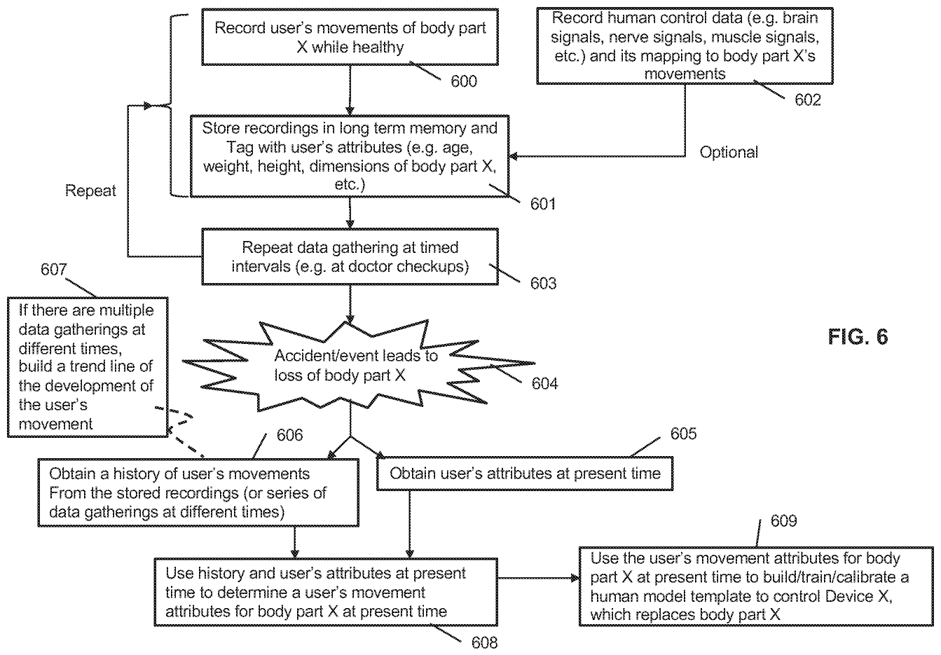

FIG. 6 provides a process for building, training, or calibrating a human model template for a given user based on previously collected data about the given user. While the example in FIG. 6 relates to movement of a body part and a robotic device that replaces the body part (e.g. a prosthetic limb), it can be appreciated that the process in FIG. 6 is applicable to other biological or physical aspects of a person that could be lost-then-replaced or otherwise augmented. For example, voice data of a user could be recorded, and the device is a robotic voice box (e.g. an audio speaker with voice intelligence). For example, sight data of a user could be recorded, and the device is an intelligent artificial vision system connected to user's brain.

Block 600: The process includes recording the user's movements of body part X while that body part is healthy. For example, the user movements can be recorded using a camera, radar, infrared, motion sensors, an inertial measurement unit, etc.

Block 601: The system stores the recordings in long term memory and tag these recording with the user's attributes (e.g. age, weight, height, dimensions of body part X, etc.) at the time of the recording.

Block 602: Optionally, the system also simultaneously records human control data (e.g. brain signals, nerve signals, muscle signals, etc.) while the block 600 takes place, so that the human control data can be mapped to body part X's movements. This information is also stored in the recording in block 601. It will be appreciated that, if voice data is being recorded, the voice data and the human control data are mapped together. It will be appreciated that, if sight data is being recorded, the sight data and the human control data are mapped together.

Block 603: Repeat the data gathering at timed intervals (e.g. at doctor checkups).

Block 604: An accident or some other event leads to loss of body part X.

Block 605: The system obtains the user's attributes at the present time.

Block 606: The system obtains a history of user's movements from the stored recordings (or series of data gatherings at different times),

For example, as per block 607, if there are multiple data gatherings at different times, the system computes a trend line of the development of the user's movement.

Block 608: The system uses the obtained history and user's attributes at the present time to determine a user's movement attributes for body part X at the present time. For example, this could be computed by using trend fitting algorithms, prediction algorithms (e.g. artificial intelligence), and regression computations.

Block 609: The system uses the user's movement attributes for body part X at the present time to build or train or calibrate a human model template to control Device X, which replaces body part X.

In this way, Device X should move in the same way, or have certain functions in the same way, as body part X.

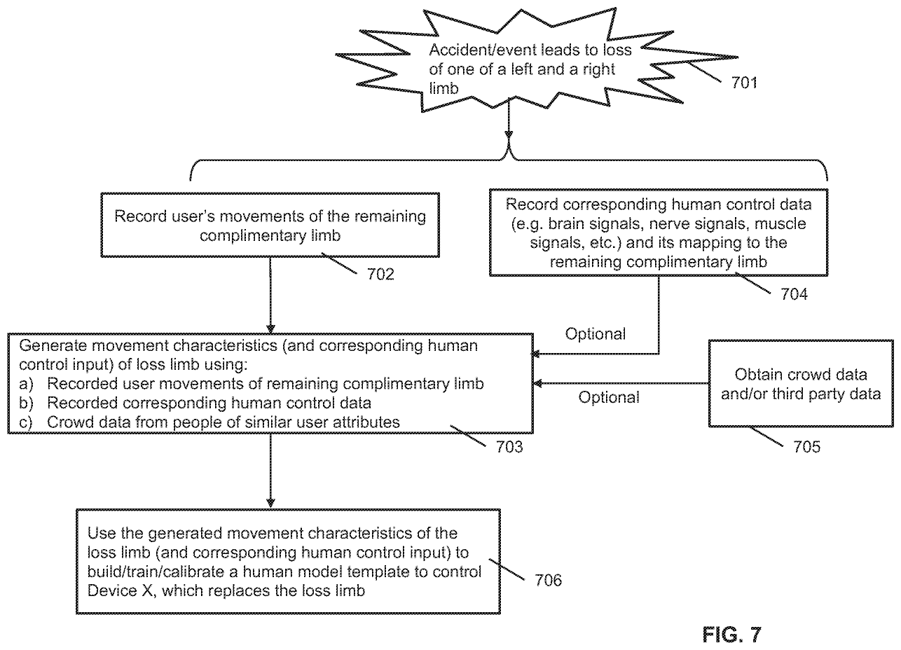

FIG. 7 shows an example process in which one of a right limb and a left limb on a person is removed and the remaining limb is used to train a human template model for a robotic limb that replaces the removed limb.

Block 701: An accident or event leads to loss of one a left and a right limb.

Block 702: The system records user's movements of the remaining complimentary limb.

Block 704: Optionally, the system records the corresponding human control data (e.g. brain signals, nerve signals, muscle signals, etc.) and its mapping to the remaining complimentary limb. In an example embodiment, if the recording of the human control data is for a remaining right arm, then the system uses this human control data to synthesize new human control data for the left arm that has been lost.

Block 703: The system generates movement characteristics (and corresponding human control input) of loss limb using: (a) recorded user movements of the remaining complimentary limb; (b) optionally, the recorded corresponding human control data; and (c) optionally, crowd data from people of similar user attributes 705 are used to generate movement characteristics of the loss limb. It will be appreciated that if the recorded user movements are for the remaining right arm, then the system transforms the recorded user movement data into movement data of a left arm, such as be flipping orientations of positioning and force vectors. In this way, the recorded user movements of the remaining right arm can be used to train a robotic prosthetic left arm.

In an example embodiment, the data (a), (b) and (c) are all blended together to generate movement of the loss limb. These different types of data can be weighted higher and lower relative to each other.

Block 706: The system uses the generated movement characteristics of the loss limb (and corresponding human control input if available) to build or train or calibrate a human model template to control Device X, which replaces the loss limb.

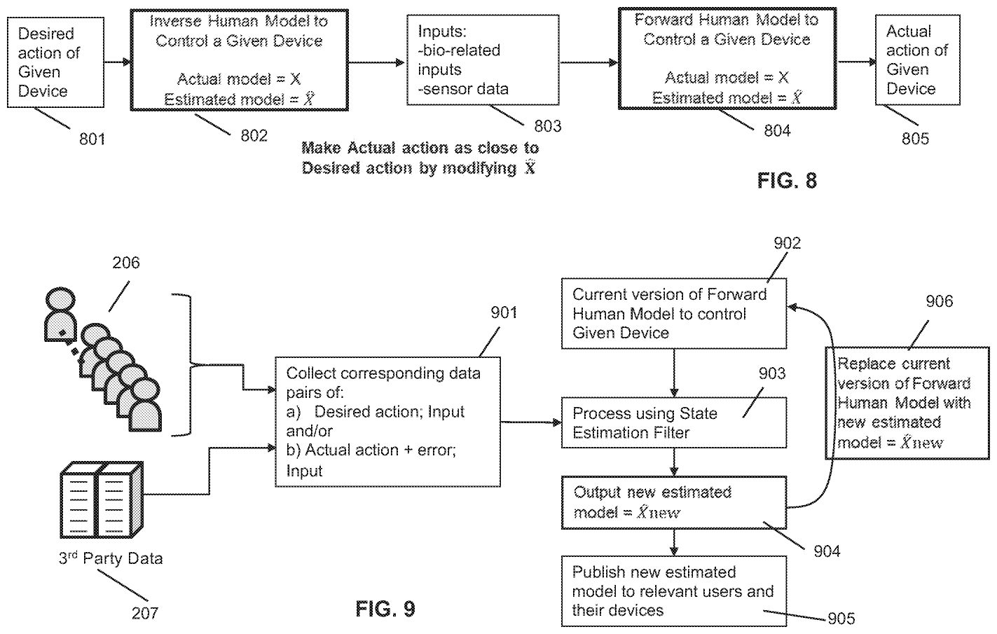

The following discussion relates to using crowd data from the human node edge network to collaboratively update the human model templates in the library. Examples of different processes are shown in FIGS. 8 to 16.

FIG. 8 shows the interaction between a forward human model to control a given device 804 and a corresponding inverse human model to control the same given device 802. A desired action of the given device 801 is inputted into the inverse human model 802, which then accordingly outputs values 803 (e.g. bio-related inputs, sensor data, etc.). These values 803 are inputted into the forward human model to control the given device 804, which accordingly outputs the actual action of the given device 805. Both the inverse human model 802 and the forward human model 804 have the same model parameters X, which can be estimated by estimated model parameters 1. The forward human model 804 and, accordingly, the inverse human model 802 are accurate when the actual action of the device 805 matches the inputted desired action of the device 801.

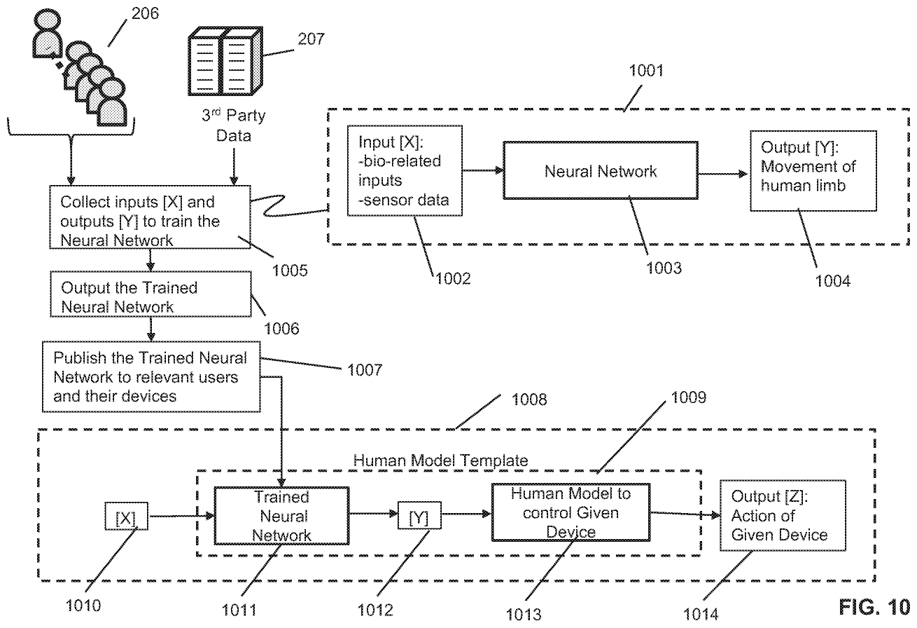

In this context, FIG. 9 provides a process use the crowd data 206 or third party data 207, or both, to compute estimated model parameters \hat{X} that drive the actual action of the given device 805 to match the desired action of the given device 801.

Block 901: The system collects corresponding data pairs of a desired action and an input. In addition or the alternative, the system collects corresponding data pairs of an actual action with the associated error, and an input. In particular, actual action+error=desired action. This data is obtained from one or both of the data sources 206 and 207, and could be pre-processed in order to form the data pairs.

Block 903: The system processes, using a state estimation filter, these data pairs and a current version of the forward human model to control the given device 902. The state estimation filter estimates the parameters of the forward human model. Examples of state estimation filters include Kalman filters, extended Kalman filters, unscented Kalman filters and particle filters.

Block 904: The system outputs the new estimated model, and in particular \hat{X} new.

Block 905: The system publishes the new estimated model to relevant users and their devices.

Block 906: The system replaces the current version of the human model with the new estimated model containing \hat{X} new.

The above process is ongoing and continuous as more crowd data and 3^{rd} party data becomes available to form more data pairs.

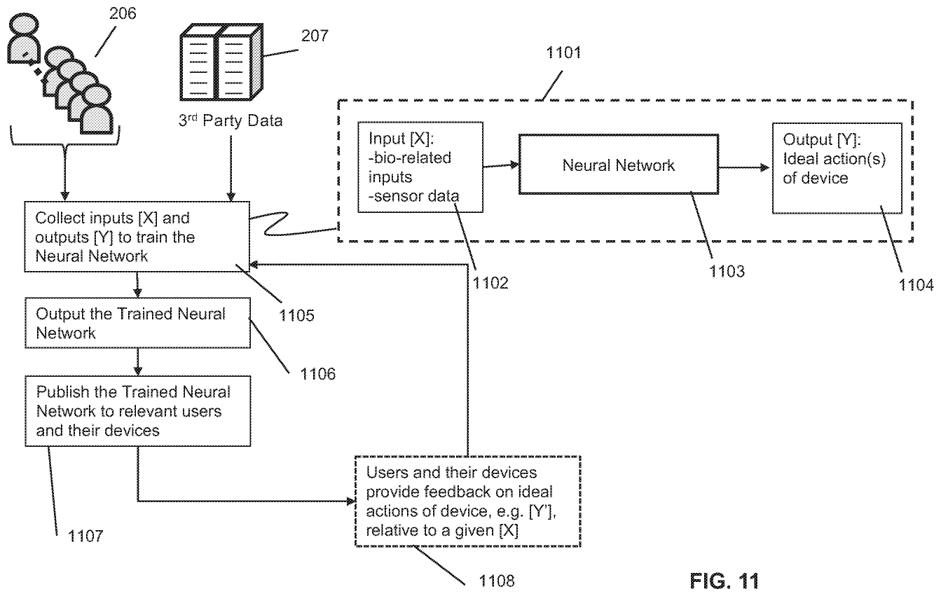

Turning to FIG. 10, another approach that uses neural networks is provided for adapting crowd data to update a human model template.

This approach recognizes that obtaining bio-related data to control a healthy limb is relatively easy. This approach also recognizes that training a robotic device to mimic a healthy limb is relatively easy. It is also herein recognized that it can be relatively more difficult to map bio-related data to control a robotic device.

A computational model 1001 includes a neural network 1003 that is trained based on inputs [X] 1002 of bio-related data or sensors data, or both, and on outputs [Y] 1004 of the movement of a human limb.

Block 1005: In order to train the neural network 1003, data from the collaborative human node edge network 206 and third party data 207 are collected. The system uses this collected data to train the neural network 1003. In an example embodiment, this collection and training process can be ongoing and continuous from a stream of crowd data and 3rd party data.

Block 1006: The system outputs the trained neural network.

Block 1007: The system publishes or transmits the trained neural network to the one or more relevant users and their devices. This publishing operation could be done when conditions are met, or at certain time intervals, or both.

The computational model 1008 shows a human model template 1009 that uses the trained neural network 1011.

In particular, input(s) [X] 1010 are inputted into the trained neural network 1011, which outputs the movements of a human limb [Y]. This is based on the computational model 1001.

The outputs [Y] are then used as inputs to the human model to control a given device 1013. This human model 1013 accordingly outputs the commands or signals [Z] to control the actions of the given device.

As can be seen, the human model template 1009 includes computations for both the trained neural network 1011 and the human model to control the given device 1013. This is on the basis that the human model to control the given device 1013 takes the movement of a human limb as input, and is accurately trained or developed since the relevant data is readily available.

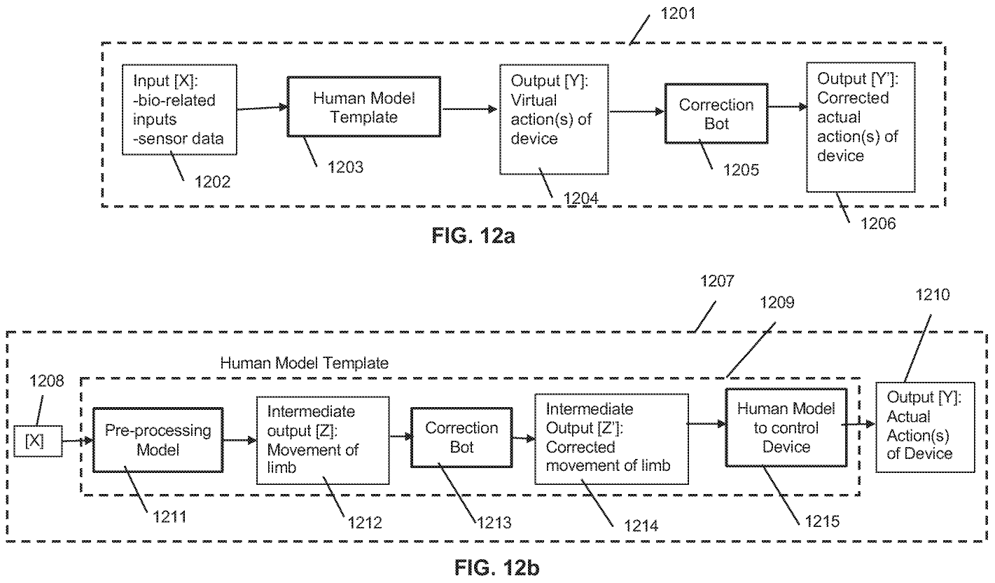

FIG. 11 is another approach that uses a neural network to adapt crowd data to update a human template model. The computational model 1101 feeds inputs [X] 1102, which could be bio-related data and other sensor data, into a neural network 1103. The neural network in turn is trained to output the ideal action(s) of a given device [Y] 1104.

It will be appreciated that the neural network 1103 can form part of the computations of the human model template along with other computations (e.g. pre-processing and post-processing computations), or forms the entire set of computations of the human model template.

Block 1105: The system collects inputs [X] and outputs [Y] from the data sources 206, 207 and uses this collected data to train the neural network 1103.

Block 1106: The system outputs the trained neural network.

Block 1107: The system publishes the trained neural network to the relevant users and their devices.

Block 1108: Users and their devices (e.g. also called human edge nodes) provide feedback on ideal actions of a device (e.g. called [Y′]) relative to a given [X]. These new pairings of [X] and [Y′] are collected (block 1105) and used to retrain the neural network. In this way, as more data becomes available, the neural network becomes more accurate in taking inputs to output the actions of the devices as intended by the user.

For example, users or their devices, or both, have a controller to identify the desired or ideal action [Y′] relative to a given [X]. For example, in the field of robotics, a user can physical grasp and manipulate a robotic limb to show the ideal movement of that limb (i.e. [Y′]) that was intended for a given input [X].

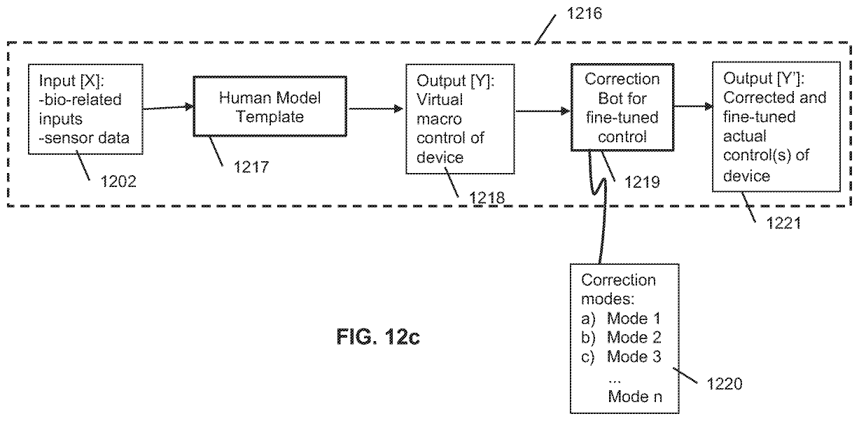

FIGS. 12a, 12b and 12c show other computational models that each have a correction bot. The purpose of the correction bot is to apply artificial intelligence based on third party data to correct the outputs of models. In other words, the correction bot can be combined with other human model templates.

In FIG. 12a, the computational model 1201 includes a human model template 1203 and a correction bot 1205. The human model template 1203 receives input [X] 1202, which could be bio-related inputs or sensor data, or both, and then processes the input [X] to generate an intermediate output [Y] 1204. The intermediate output [Y] 1204 in this context is a virtual action or set of virtual actions of the device. The actions are virtual since they are not actually acted upon by the device, since these virtual actions are used as input into the correction bot 1205. In particular, the correction bot 1205 processes the intermediate output [Y] to generate a corrected output [Y′] 1206, which is a corrected action or actions that are actually implemented by the device.

In FIG. 12b, the computation model 1207 includes a pre-processing model that takes bio-related data and other sensor data as inputs [X] 1208, and then outputs an intermediate output [Z] 1212, which is a movement of a limb. For example, the pre-processing model 1211 is similar to the approach of the trained neural network 1011 described in FIG. 10, which outputs the movement of a limb so that a human model 1013 can use this limb movement data as input.

However, in FIG. 12b, the intermediate output [Z] is used as input by the correction bot 1213 to generate a corrected intermediate output [Z′] 1214, which is data representing a corrected movement of the limb. This corrected intermediate output [Z′] is then inputted into the human model to control a device 1215, which in turn generates an output [Y] 1210. The output [Y] is the set of data or commands that cause the actual action or actions of the device. The human model 1215 is, for example, similar to the human model 1013 described in FIG. 10.

FIG. 12c shows a computational model 1216 that is similar to the computational model 1201 in FIG. 12a. The inputs [X] are inputted into the human model template 1217, which outputs virtual macro control data [Y] for the device 1218. The template 1217 purposely computes macro controls, as they are intended to be further processed by the correction body 1219 to fine-tune the outputs. In an example aspect, the correction bot 1219 applies one or more correction modes that is/are selected from a library of correction modes 1220. In an example embodiment, the correction mode can be based on skill level (e.g. mode 1 is a basic skill level; mode 2 is an intermediate skill level; and mode 3 is an expert skill level). In another example embodiment, the correction modes are specific to certain contexts (e.g. mode 1 is for playing a musical instrument; mode 2 is for playing sports; and mode 3 is for activities of daily living, like dressing, eating and bathing). In another example embodiment, the correction modes are specific to certain experts (e.g. mode 1 is for playing basketball like Stephen Curry; mode 2 is for playing basketball like Michael Jordan; mode 3 is for playing basketball like LeBron James; etc.). In an example where the device is a robotic arm for playing music, a combination of modes include: a mode for playing music; an add-on mode for playing classical music style; and an add-on for playing melancholy music. It can be appreciated that the correction modes can be varied to compute fine-tuned outputs [Y′] 1221 that are used to actually control the device.

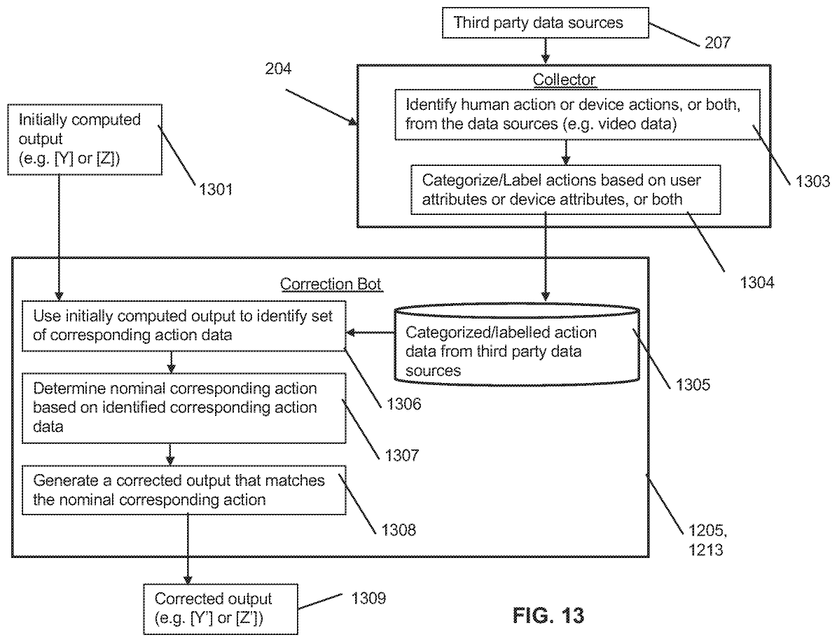

FIG. 13 shows example executable computations of a correction bot 1205, 1213 that outputs a corrected output. In particular, the correction bot 1205 outputs the corrected output [Y′] as per the embodiment in FIG. 12a, and the correction bot 1213 outputs the corrected output [Z′] as per the embodiment in FIG. 12b.

The collector 204 collects data from third party data sources 207, such as from the Internet. As per block 1303, the collector identifies human action or device actions, or both, from the data sources. The data could be in the form of video, audio data, machine data, social network data, or text data, or a blend of different types of data. For example, data from online video databases like YouTube, FaceBook, Vimeo, etc. provide large amounts of data that could be mined to identify human actions or device actions, or both. At block 1304, the collector categorizes or labels these actions based on user attributes or devices attributes, or both. This categorized or labelled action data is saved in a database 1305 that is accessible to the correction bot.

Independently, the correction bot receives initially computed outputs 1301 (e.g. [Y] as per FIG. 12a and [Z] as per FIG. 12b). At block 1306, the correction bot uses the initially computed outputs to identify sets of corresponding action data from the database 1305.

In particular, the correction bot uses machine learning to identify the action data in the database 1305 that most closely matches the initial output data 1301.

In an example computation, the correction bot identifies the action data from the database 1305 based on common user attributes or common device attributes, or both, between the given human edge node and the labels applied to the data in the database 1305. This produces a set of filtered action data from the database 1305. After this initial filtering, the correction bot computes a similarity matching between the initially computed outputs (e.g. [Y] or [Z]) and the filtered action data from the database. It can be appreciated that other data science approaches can be used to identify the set of action data from the database 1305 that corresponds to the data 1301.

At block 1307, the collection bot determines a nominal corresponding action based on the identified corresponding action data. This can be based on various data science computations, such as averaging, clustering, K-means, etc. In the context of the example embodiment in FIG. 12c, the collection bot determines the nominal corresponding action based on the automatically applied correction mode.

At block 1308, the collection bot generates a corrected output 1309 that matches the nominal corresponding action.

This process in FIG. 13 identifies the true intent of the user based on norms and big data, and then corrects the output to match the true intent. The norms and the big data are expansive and readily available (e.g. Internet data) compared to bio-related data.

For example, video data on YouTube includes thousands of videos of males of a certain demographic moving his arms in various contexts. This video data could be analyzed to establish nominal movement, which is used to inform the correction bot of the truly intended device action by a given male (i.e. a human edge node) of the same certain demographic.

In another example, audio data on podcasts includes thousands of audio recordings of females of a certain demographic speaking in various contexts. This audio data could be analyzed to establish nominal speech patterns, which is used to inform the correction bot of the truly intended speech device action by a given female user (i.e. a human edge node) of the same certain demographic.

The system also uses the corrected mappings between the input and the corrected outputs to update the human model templates in the library (e.g. by a librarian bot), or at a given human edge node (e.g. by a personal bot), or both.

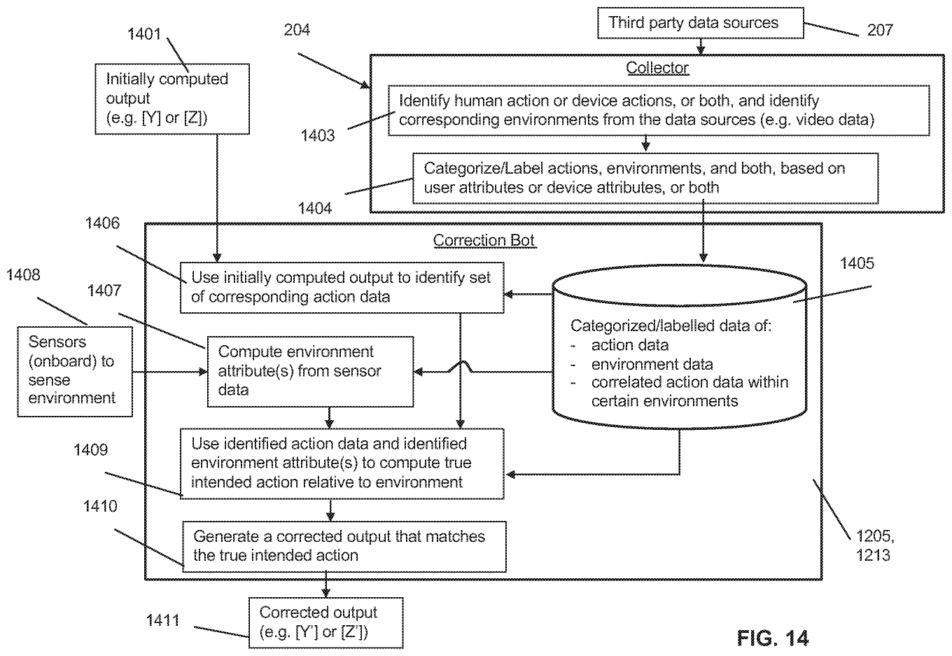

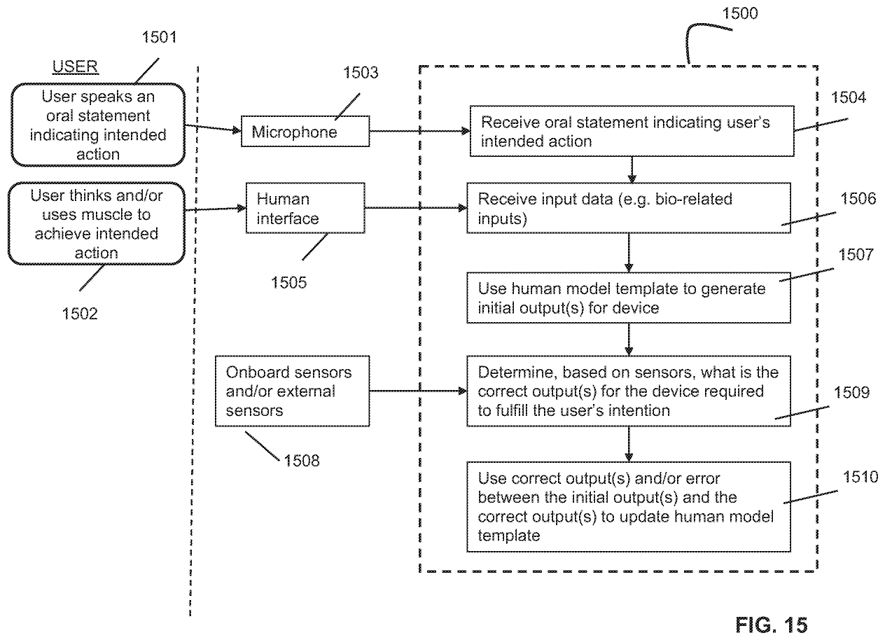

FIG. 14 shows an alternative computing process to the process shown in FIG. 13. The processing in FIG. 14 additionally takes into account sensor data that senses the environment in order to identify environmental or contextual settings of certain actions. The processing in FIG. 15 includes identifies normal or typical characteristics of actions in a given environment, on the basis that typical characteristics of actions change with different environments or contexts. Therefore, the system can more accurately identify a given human edge node's true intent by looking at big data from third party sources (e.g. Internet data) and from sensor data of the given human edge node.

Block 1403: From the data provided by the data sources 207, the collector 204 identifies human actions or device actions, or both, and identifies the corresponding environments in which the human actions of device actions take place.

Block 1404: The collector then categorizes or labels these actions, environments, and the combination thereof, based on user attributes or device attributes, or both. The user attributes or device attributes, or both, can be obtained from the metadata associated with the data, or can be identified by processing the data.

The categorized or labelled data include the action data of people or of devices, or both; the environment or contextual data; and the correlations or mappings between certain action data and certain environments or contexts. This data is labelled or tagged according to user attributes or device attributes, or both, and is stored in a database 1405.

Block 1406: Independently, the correction bot obtains the initially computed outputs (e.g. [Y] or [Z]) to identify a set of corresponding action data from the database 1405.

Block 1407: The correction bot also obtains environment or context data from one or more sensors 1408, and obtains data from the database 1405, in order to compute the presently sensed environment attributes.

The sensor or sensors 1408 sense the present environment of the device, or the user, or both. The sensor or sensors could include: camera sensors, audio sensors, RADAR, LiDAR, temperature sensors, heartrate sensors, tactile sensors, location or positioning sensors, etc. The sensors are preferably onboard the device or are with the person (e.g. mounted to the person, or on their mobile computing device). However, the sensors could be external to the device and the person. For example, the sensors could include car sensors, satellite sensors, sensors mounted in the local environment of the user, sensors from other human edge nodes in proximity to the subject user.

Block 1409: The correction bot uses the identified action data (from block 1406) and identified environment attribute(s) (from block 1407) to compute the true intended action of the user relative to sensed environment. The decision of the correction bot is reflective of the training data in the database 1405.

Block 1410: The correction bot then generates a corrected output 1411 that matches the true intended action of the user.

For example, the above correction process detects that the present environment of a robotic hand are fingers on a plano key. According to the categorized and labelled data of in the database 1405, the correction bot identifies that the typical motion for a human finger in this environment is for a finger to hit a plano key. This information is incorporated into deciding the true intention of the initially computed output 1401.

In another example, the correction bot detects that the present environment of a robotic leg is a soccer field with a soccer ball in front of the robotic leg. According to the categorized and labelled data of in the database 1405, the correction bot identifies that the typical motion for a human leg in this environment is for the leg to kick the soccer ball. This information is incorporated into deciding the true intention of the initially computed output 1401.

In another example, the correction bot detects that the present environment of a robotic hand is kitten in contact with the robotic hand and that the kitten is on the lap of the user (who is operating the robotic hand via a human-computer interface). According to the categorized and labelled data of in the database 1405, the correction bot identifies that the typical motion for a human hand in this environment is to move the hands and fingers gently through the fur to pet the kitten. This information is incorporated into deciding the true intention of the initially computed output 1401.

The system also uses the corrected mappings between the input and the corrected outputs to update the human model templates in the library (e.g. by a librarian bot), or at a given human edge node (e.g. by a personal bot), or both.

FIG. 15 provides a process for using oral data of a user for labelling data, which in turn could be used for training, correction and calibration of human model templates. The labelling of data could also be used to help populate a categorized or labelled database (e.g. databases 1305, 1405).

Using the process of FIG. 15, a user orally calls out their intended actions in advance in order to label the intended action. For example, if a user with a robotic arm intends to grasp a soda can, the user will first speak out “I am going to pick up the soda can”. The user then proceeds to use their body (e.g. brain signals, nerve signals, muscle signals, chemical signals, etc.) to attempt to control a device to perform the intended action. This voice data is processed to generate an intention label, which can be subsequently associated with bio-related data (e.g. input data) and outputted data from a given human model template. The combination of these different data can be used to build, calibrate, train, or modify human model templates.

To capture the voice data, an audio sensor 1503, like a microphone, is incorporated into the device, or is external to the device. For example, the microphone is on the user's mobile device or some other device. Below are details of an example oral labelling process 1500.

Block 1501: The user speaks an oral statement indicating intended action. This is recorded by an audio sensor 1503.

Block 1504: The system receives the oral statement (e.g. data derived from the recorded audio data) indicating the user's intended action.

In other example aspects, in alternative to 1501, 1503, 1504, other approaches are used to explicitly capture the user's intended action. For example, a user can type in their action, or a user can select their intended action from a pre-determined list of actions, or some other approach, and these explicit intention labels are associated with the bio-related data (e.g. the input data).

Block 1502: The user then thinks and/or uses their muscle to achieve the intended action of the device. This bio-related input is recorded by the human computer interface 1505.

Block 1506: The system receives this input data.

Block 1507: The system uses a human model template to generate initial output data to control the device.

Block 1509: The system determines, based on sensor data, what is the correct output action for the device required to fulfill the user's intention. The user's intention is based on the received oral statement.

It could be appreciated that the sensor data is measurable from sensors onboard the device, or external to the device 1506. The sensors, for example, could record various types of data, including images, video, audio, temperature, user heartrate, location or position, RADAR, LiDAR, etc.

Block 1510: The system uses the determined correct output(s), or a computed error between the initial output(s) and the correct output(s), to update the human model template.

In an example embodiment, these initial output(s) to a device could lead to a virtual action of a device as computed in a virtual environment that corresponds to (e.g. is a digital model of) the real world environment. These virtual inputs are then corrected, leading to a corrected actual action of the device. In this way, the initial output(s), which is not correct, is never realized in the real world environment and, instead, the correction outputs of the device are only realized in the real world environment.

In another example, the initial output(s) are actual actions taking place in the real world environment, and the corrected outputs are then subsequently implemented to adjust the actual actions of the device.

In an example scenario that uses the above process, a user speaks out: “I am going to grab that soda can.” The user then uses their thoughts (e.g. brain signals) to try and control their robotic prosthetic arm to grab the soda can. The system then uses these brain signals to compute the initial outputs for the robotic prosthetic arm to grab the soda can, according to the human model template. One or more cameras or one or more radar sensors, or both, which are mounted to the robotic prosthetic arm, sense the location of the soda can relative to a position (e.g. an actual position or a virtual position) of the robotic prosthetic arm based on the initial outputs. The system then uses this relative positioning (e.g. which is an error between the initial outputs for the device and the position of the soda can) to compute a correct path for moving the end of the robotic prosthetic arm to grab the soda can; this is in alignment with the user's spoken intention. From this path, the system computes the correct outputs for the movement of the prosthetic arm to grab the soda can. In other words, during this process, the system now has the following training data: true intention data (e.g. ideal outputs), recorded user intention data (e.g. bio-related inputs), and corrected output data in relation to the human model template that aligns with the true intention data. This data can be used to build, train, calibrate or modify human model templates.

This process is preferably repeated across many users in order generate volumes of data to calibrate human model templates.

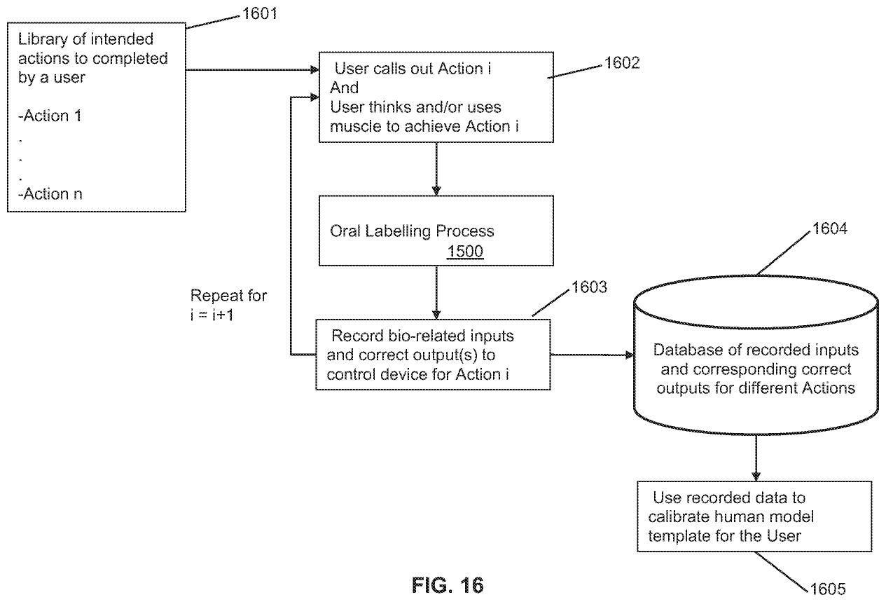

Turning to FIG. 16, the oral labelling process 1500 is used in different action scenarios for a given user in order to calibrate a human model template for the given user.

A library 1601 of intended actions are presented to the user, for example, either orally or through a display device. The library includes a listing of actions (e.g. Action 1, . . . , Action n) to be completed by the user.

Block 1602: The user calls out Action i (e.g. speaks out Action i), or otherwise the system commands the user to perform Action i. The user thinks or uses muscles, or both, to achieve Action i. The value i is an integer identifying a given action in the library 1601, and it is initially set at i=1.

The oral labelling process 1500 is the performed using Action i.

Block 1603: The system records the bio-related inputs and the correct outputs to control the device to achieve Action i.

The process iterates for i=i+1.

In this way, the system collects data pairs of recorded inputs (e.g. bio-related signals) and corresponding correct outputs that are labelled for different actions. This data is stored in the database 1604.

Block 1605: The system uses this recorded data to calibrate a human model template for the given user.

The list of actions in the library 1601 is preferably diverse and cross-correlated, so that, after completing the actions in the library 1601, the human model template is calibrated for the given user for many different actions that are not necessarily listed in the library 1601.

As one example listing of actions for a robotic hand, the actions in the library 1601 can include: shake hands; write your name; draw a circle; draw a square; pour water into a cup; fold a piece of paper; shovel sand; and catch a ball.

It will be appreciated that there are multiple approaches to adapting crowd data of from the human edge node network and third party data to update the human model templates. These updates are, for example, ongoing. The approaches described above can be combined together in various ways. Other currently known and future known computational approaches can be used with the system described herein to update the human model templates.

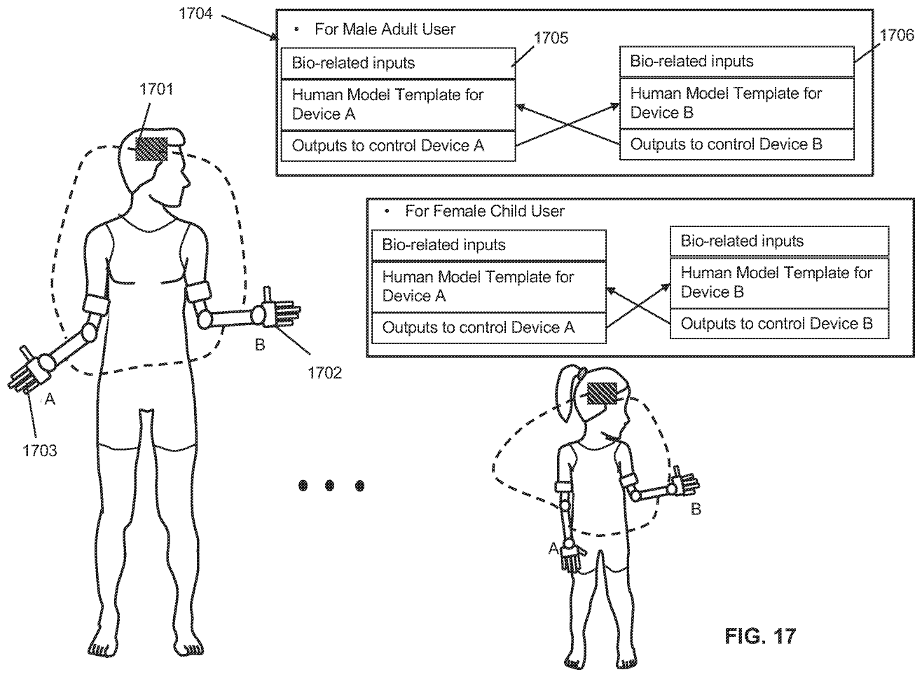

Turning to FIGS. 17 to 24, the following is a discussion of different example combinations of human-computer interfaces and devices. The principles describes in these example combinations can be applied to other combinations of human-computer interfaces and devices.

FIG. 17 shows an adult male that has a right robotic arm 1703 (e.g. Device A) and a left robotic arm 1702 (e.g. Device B) that are each controlled by a brain-computing interface 1701, or another type of human-computing interface. The system includes that these different devices 1702, 1703 have device-to-device intelligence, so that the right robotic arm does know what the left robotic arm is doing at the device level, independent of the user's thoughts. In other words, the left robotic arm and the right robotic arm coordinate with each other so that they do not crash, or that they work more efficiently and effectively together.

In an example embodiment, each robotic arm 1702, 1703 is equipped with its own communication device, and these communication devices can directly communicate with each other.

In another example embodiment, each robotic arm 1702, 1703 is equipped with its own one or more sensors and own one or more onboard processors, so that each robotic arm can detect the movement, position, or action of the other robotic arm.

The ability for the robotic arms 1702, 1703 to sense each other or to communicate directly with each other is shown by the dotted line therebetween.

The dotted lines from the brain-computing interface 1701 to the robotic arms 1702, 1703 indicate the interface's ability to communicate with the arms 1702, 1703.

A multi-device human model template 1704 is provided that includes a human model template 1705 for controlling the right robotic arm 1703 and a human model template 1706 for controlling the left robotic arm 1702, and these human model templates 1705, 1706 interact with each other.

In an example embodiment, the computed outputs to control the right robotic arm from the human model template 1705, are used as additional inputs to the human model template 1706 for the left robotic arm. Conversely, the computed outputs to control the left robotic arm from the human model template 1706, are used as additional inputs to the human model template 1705 for the right robotic arm.

Similar models are adapted for other people having other user attributes, such as a female child user with two robotic arms. The multi-device human model template for a female child user would be different than the human model template 1704 for the male adult user.

It will be appreciated that the principles of the multi-device human model template can be applied to control multiple devices at the same time by a user via a human-computer interface. For example, a user could control all four robotic limbs (e.g. two legs and two arms). In another example, the user controls three robotic drones flying in formation through a human-computer interface. In another example, the user controls four robotic surgical arms using a human-computer interface. In another example, the user controls two or more different types of devices using a human computer interface. For example, two different types of devices could be (1) a virtual avatar that can be displayed in various ways, such as on a display screen, on a head set, on a projector, etc.; and (2) a voice bot or speech device, so that the virtual avatar moves around and verbally talks based on the thoughts of the user.

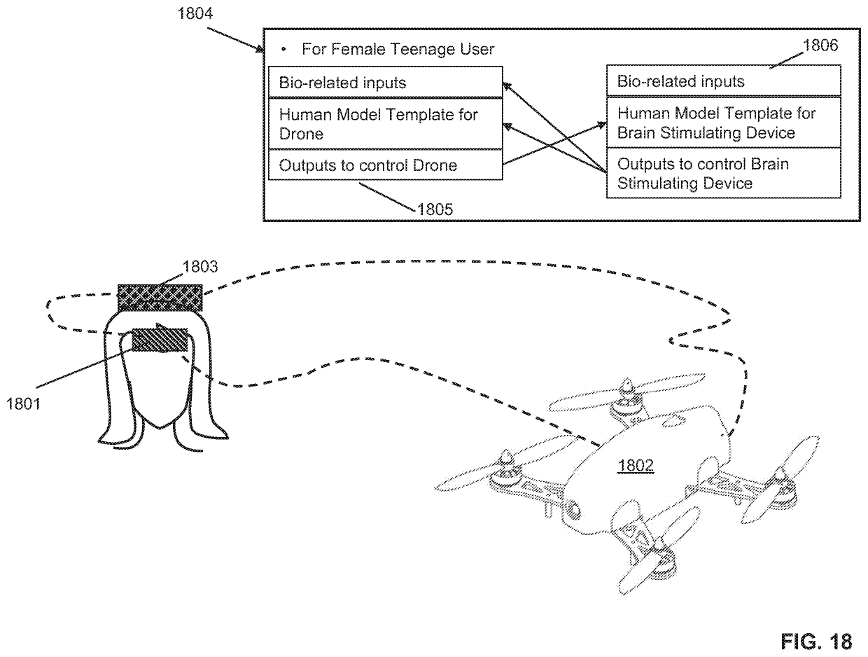

FIG. 18 shows a female teenage user equipped with a brain-computing interface 1801, or another type of human-computing interface. The user, for example, uses her thoughts to control a drone aircraft 1802. The user is also equipped with a brain stimulating device 1803 that augments the user's ability to control the drone 1802 using her thoughts.

For example, the brain stimulating device improves the user's reaction time to generate brain signals, or increasing the brain's state of plasticity, or increases the speed at which new neural pathways are made, or increases brain signal strength, or reduces brain signals that are not relevant to controlling the drone 1802, or a combination thereof.

Examples of brain stimulating devices include cognitive enhancement devices (CEDs). Examples of CEDs include transcranial direct current stimulators, transcranial magnetic stimulators, cranial electrotherapy stimulators, and neurofeedback equipment. For example, transcranial direct current stimulators send a small direct current between two or more electrodes to facilitate or inhibit spontaneous neuronal activity. Transcranial magnetic stimulation is a neurostimulation and neuromodulation technique that uses electromagnetic fields to penetrate the scalp and skull. Cranial electrotherapy stimulators apply pulsed, alternating microcurrent transcutaneously to the head via electrodes placed on the earlobes. Neurofeedback equipment uses realtime displays of electrical patterns from brainwave activity to regulate or suppress different patterns of activity. In an example embodiment, CEDs can be used to adjust the state of the brain so that less neuron inputs are required for a given neuron to reach an electrical threshold, so that the given neuron can more responsively reach the electrical threshold to fire an action potential.

As shown in FIG. 18, the devices 1803, 1801 and 1802 are all in communication with each other. For example, these devices are in communication with each other over a wireless network. The device-to-device intelligence coordinates the actions of these devices to collaboratively improve the user's ability to control the drone using her thoughts.

A multi-device human model template 1804 for the user is shown, which includes a human model template 1805 for controlling the drone 1802 using the bio-related inputs of the user, and a human model template 1806 for controlling the brain stimulating device 1803 using the bio-related inputs of the user.

In an example embodiment, the brain stimulating device 1803 is not consciously controlled by the user, but the brain stimulating device 1803 uses inputs from the brain-computing interface 1801 and information about the drone 1802 to control how it stimulates the brain.

As per the template 1804, outputs from the template 1805 to control the drone are used as inputs to the template 1806 to control the brain stimulating device. In turn, the outputs from the template 1806 to control the brain stimulating device biologically affect the bio-related inputs (e.g. the brain operation) to the template 1805. The outputs from the template 1806 to control the brain stimulating device are also used as data inputs into the human model template 1804 for controlling the drone.

In another example aspect, there is other sensor data (e.g. environmental data or contextual data) that is processed as additional inputs by the human model template 1806 for the brain stimulating device and the human model template 1805 for the drone.

In another embodiment, there is no feedback from the drone outputs to the brain stimulating device.

It will be appreciated that the human model templates may vary based on different users.

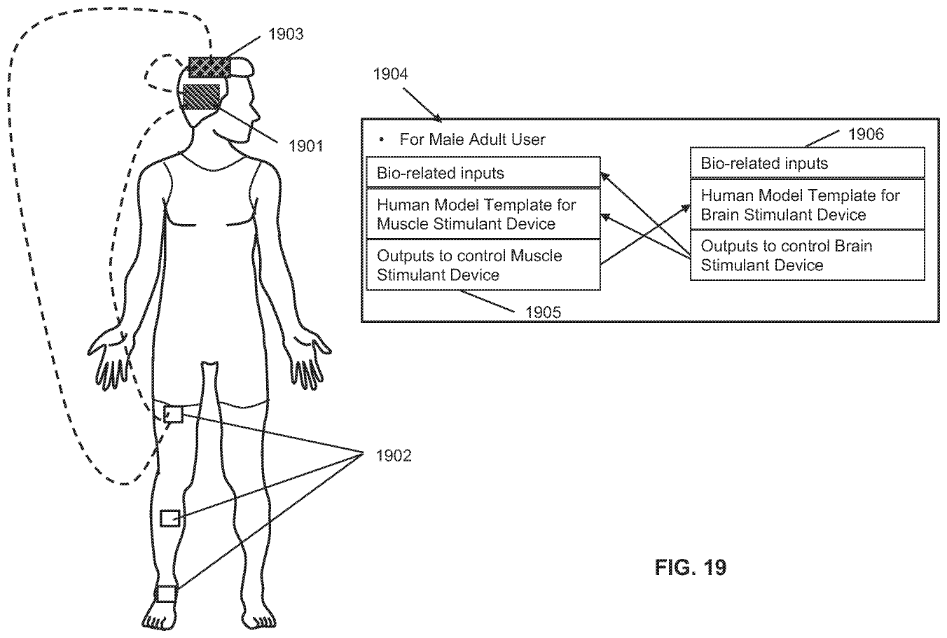

Turning to FIG. 19, a male adult user is equipped with a brain-computing interface 1901 that is used to control one or more muscle stimulating device 1902.

Examples of muscle stimulating devices are electrical muscle stimulators (EMS), also known as neuromuscular electrical stimulation (NMES) or electromyostimulation. These devices can be used to elicit a muscle contraction using electric impulses. These device can be applied to help with physical rehabilitation, help paraplegic patients move again, help athletes improve their strength, and help healthy people move faster (e.g. increase muscle reflexes). These devices can be in the form of patches applied on the skin, or electrical devices embedded on the muscle, or electrical devices embedded within the muscle.

The user is also equipped with a brain stimulating device 1903, which was described earlier with respect to the device 1803 in FIG. 18.

In the example shown in FIG. 19, using device-to-device intelligence, the brain stimulant device augments the user's brain waves (e.g. the bio-related inputs) to control the muscle stimulant device.

The multi-device human model template 1904 includes a human model template 1905 for controlling the muscle stimulating device using the user's thoughts, and a human model template 1906 for controlling the brain stimulant device. The brain stimulant device 1906 is not consciously controlled by the user's thoughts, but it can dynamically adjust its outputs to accommodate for one or more of (1) the user's thoughts (e.g. bio-related inputs) and (2) the outputs from the human model template 1904 to control the muscle stimulant device.

The human model template 1904 processes the bio-related inputs and the outputs from the human model template 1906 for controlling the brain stimulant device.

In another embodiment, the muscle stimulant device output is not fed back to the human model template 1906 for controlling the brain stimulating device.

In another example aspect, there is other sensor data (e.g. environmental data or contextual data) that is processed as additional inputs by the human model templates 1905 or 1906, or both.

It will be appreciated that the human model templates may vary based on different users.

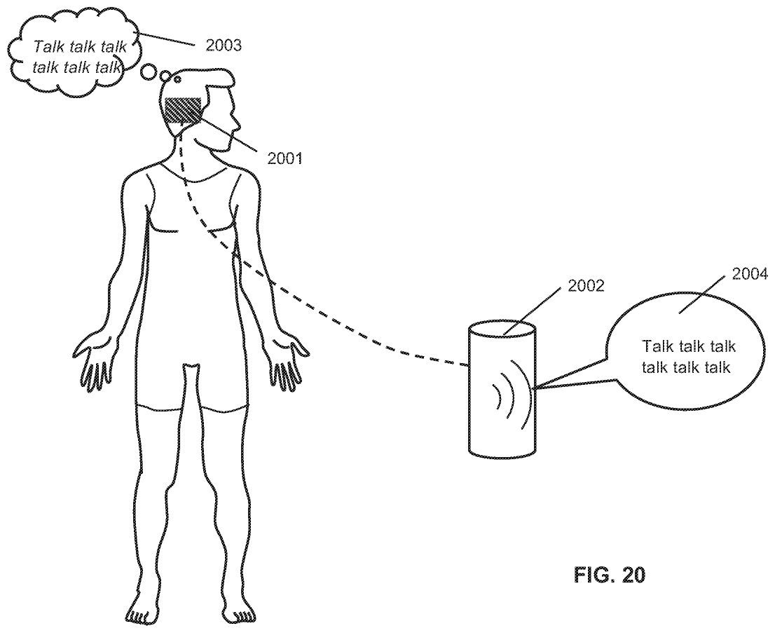

FIG. 20 shows another example embodiment of a human edge node, which includes a person equipped with a brain-computing interface 2001 to interact with a voice bot or some other speech device 2002. In particular, the interface 2001 is able to detect the brain signals while the person conducts thought speech 2003. These brain signals representing the thought speech are processed using a human model template in order to control the speech device 2002 to generate and play speech audio data 2004 that corresponds to the person's thought speech.

The term “thought speech” is herein referred to as the thoughts of a user expressed in language. For example, a person may talk to themselves within their minds, without any physical speech (e.g. no movement of the mouth, no sound); this is also known as a form of private speech. Thought speech goes beyond private speech, since it can be processed using brain-computer interfaces to communicate with others.

In other words, the user's inner thoughts are converted to digital audio speech for others to hear. This is useful, for example, if a person has lost the ability to speak. They can use the interface 2001 and the device 2002 to verbally communicate with others.

In an example embodiment, a person may have injured their voice box and can no longer speak. In another example, a person may have lost their ability to control their muscles used to speak. In another example, a person in a state of coma may be active in their mind, but have lost their ability to consciously speak.

In another example, the ability to transform brain signals to text or verbal speech, or both, allows people to linguistically communicate with each other using their minds.

It is appreciated that there are many different applications of using thought speech.

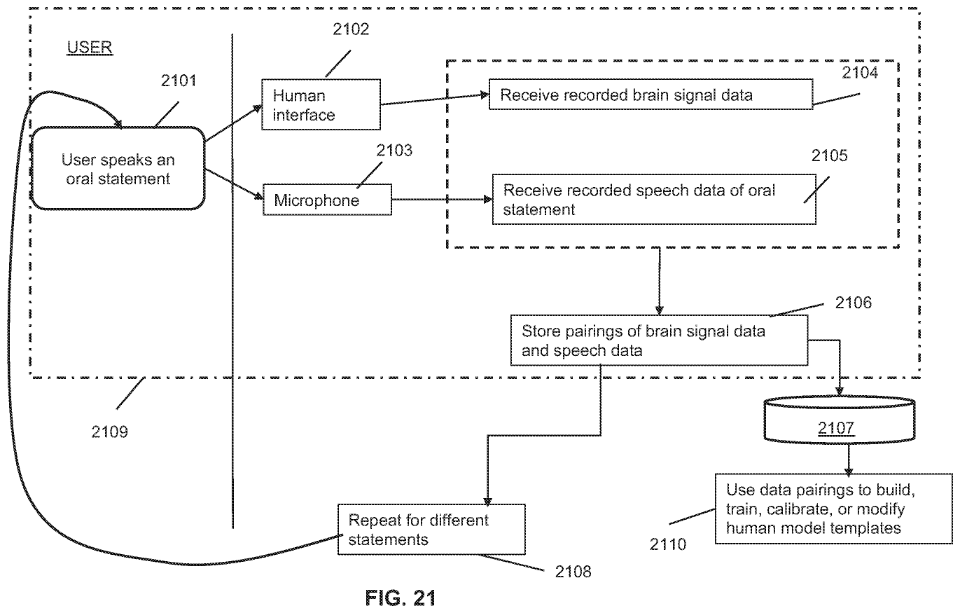

FIG. 21 provides an example of obtaining data to build, train, calibrate, or modify human model templates to convert brain signals to language data (e.g. such as speech data).

At block 2101, the user speaks an oral statement out loud. A brain-computing interface 2102 records the brain signals while this occurs, and a microphone 2103 simultaneously records the speech data. The system accordingly receives the recorded brain signal data at block 2104 and receives the recorded speech data of the oral statement at block 2105. The system stores theses pairings of the brain signal data and the speech data (block 2106) into the database 2107. The process 2109 is repeated for different statements (block 2108) in order to populate the database 2107. These data pairings are used by the system to build, train, calibrate or modify human model templates (block 2110), so that these human model templates can be used to convert brain signals representing thought speech into language data that is understandable by a computer and people.

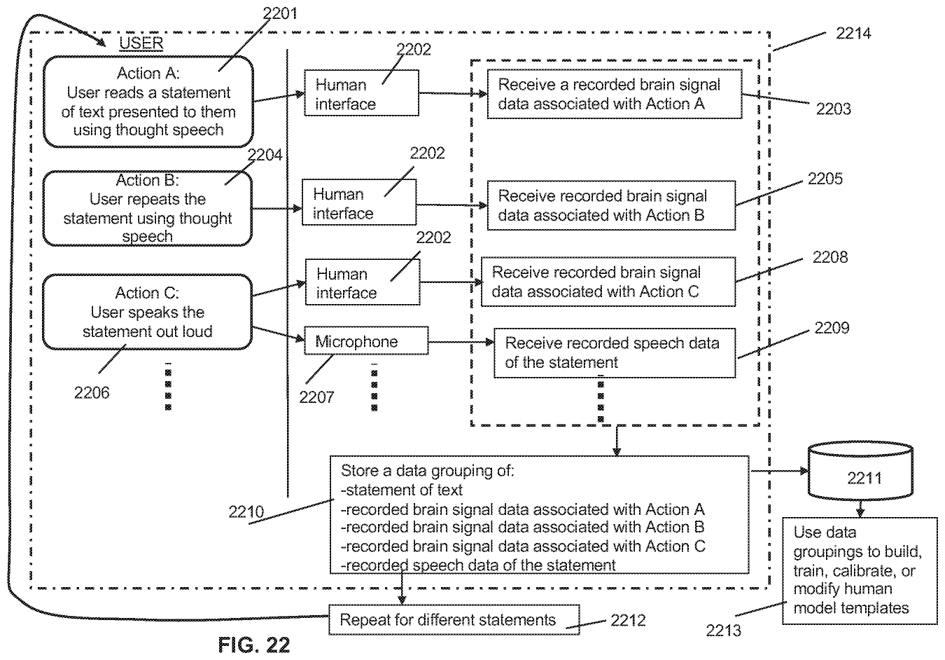

FIG. 22 provides another example of obtaining data to build, train, calibrate or modify human model templates to convert brain signals representing thought speech into language data (e.g. text, speech, etc.).

At block 2201, the user performs Action A: the user reads a statement of text presented to them using thought speech. A brain-computing interface 2202 records the brain signals while Action A occurs. At block 2203, the system receives recorded brain signal data associated with Action A.

At block 2204, the user performs Action B: the user repeats the statement in their mind only using thought speech. The brain-computing interface records the brain signals while Action B occurs. For example, Action B is done from memory and not from reading text. At block 2205, the system receives recorded brain signal data associated with Action B.

At block 2206, the user performs Action C: the user speaks the statement out loud. The brain-computing interface records the brain signals while Action C occurs and a microphone 2207 records the audio speech data also while Action C occurs. At blocks 2208 and 2209, the system receives the recorded brain signal data associated with Action C, and receives the recorded speech data of the statement.

The user could perform other actions that initiate thought speech in different circumstances. The purpose of these different actions is to find commonalities in the brain signals while thought speech occurs in different ways.

At block 2210, the system stores a data grouping of the following data in the database 2211: statement of text; recorded brain signal data associated with Action A; recorded brain signal data associated with Action B; recorded brain signal data associated with Action C; and recorded speech data of the statement.

The process 2214 is repeated for different statements (block 2212) so as to populate the database 2211 with multiple instances of data groupings, each corresponding to different statements.

At block 2213, the system uses these data groupings from the database 2211 to build, train, calibrate or modify human model templates, which convert brain signals representing thought speech into language data.

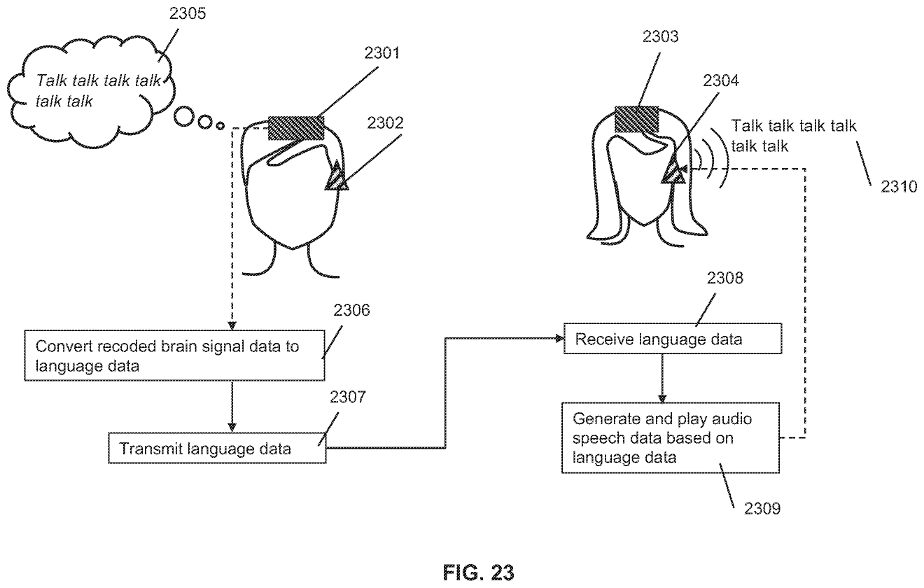

FIG. 23 shows another example application of thought speech, which facilitates thought speech conversations between users. A first user is equipped with a brain-computing interface 2301 and an auditory device 2302 (e.g. an earpiece device). The first user and their devices form a first human edge node.

A second user (i.e. another human edge node) is also equipped with a brain-computing interface 2303 and an auditory device 2304 (e.g. an earpiece device). The second user and their devices form a second human edge node.

The first user makes a statements in the form of thought speech 2305, which are brain signal data recorded by the interface 2301.

This recorded brain signal data is converted to language data (block 2306) using a human model template. This computation occurs, for example, at the first human edge node. The first human edge node transmits the language data to the second human edge node (block 2307).

The second human edge node receives the language data (block 2308) and generates and plays audio speech data based on the language data (block 2309). In other words, the audio speech data 2310 is played to the second user via their auditory device 2304, and the audio speech data 2310 matches the words expressed in the thought speech statement 2305 of the first user.

In a similar manner, the second user can use thought speech to respond to the first user. In a way, this type of conversation mimics telepathic communication.

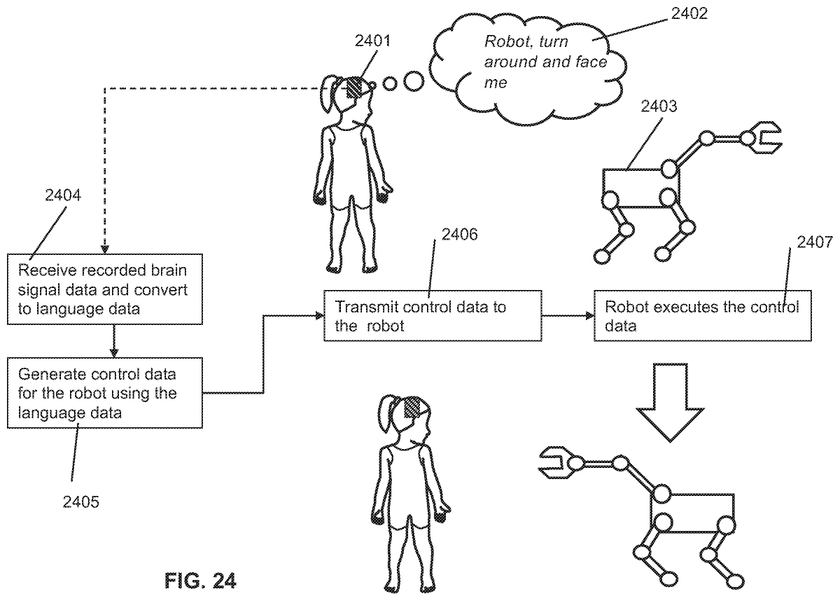

FIG. 24 applies thought speech to control a robot 2403. A user with a brain-computing interface 2401 makes a thought speech statement 2402 for controlling the robot: Robot, turn around and face me. The robot in this example is initially oriented facing away from the user. The user with their interface 2401 forms a human edge node.

The interface 2401 records the brain signals while the thought speech statement 2402 is made. At block 2404, the human edge node receives the recoded brain signal data and converts this to language data using a human model template. At block 2405, the human edge node generates control data for the robot using the language data. The processing of the language data to output control data for the robot is also completed based on a human model template. At block 2406, this control data is transmitted to the robot.

At block 2407, the robot executes the control data and the robot turns arounds to face the user.

In another example embodiment, a user uses a human-computer interface to issue a high-level command (e.g. via one or more of brain signals, eye movement, subvocalizations, micro-movements of the body, etc.) and the human-computer interface transmits the signal corresponding to the high-level command to a system of devices. The system of devices then collectively work together to determine the best way to achieve that high-level command. In other words, the user does not need to think of sub-commands, as the collective system of devices will automatically generate the sub-commands and execute the sub-commands that will execute the high-level command.

Another example application of the human model templates (not shown) includes a first user human edge node that partially controls a second user human edge node. For example, the biological signals for the first user human edge node are recorded and are used to control a device or devices attached to the second user. These devices attached to the second user control or guide the movement of the second user. In this way, a physical therapist (e.g. a first user human edge node) can guide a patient (e.g. a second user human edge node) to move in an improved manner using the thoughts of the physical therapist. This same process could be used by a coach (e.g. a first user human edge node) to guide or teach the movements of an athlete (e.g. a second user human edge node).

Another example application of the human model templates is that the user (i.e. the human edge node) is able to control a virtual bot rather than a typical conventional device. The virtual bot can be a digital avatar of a person or thing that resides in a computing system (e.g. a computing device or cloud computing system).

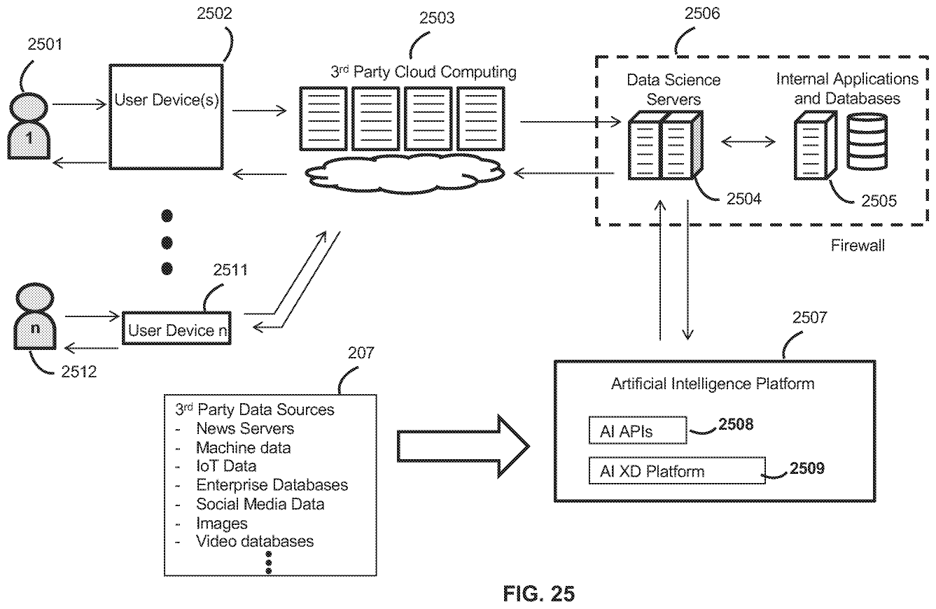

Turning to FIG. 25, an example embodiment of a computing architecture is provided for a computing platform 105 that interacts with other human edge nodes. A user device or devices 2502 interact with a user 2501. The one or more devices 2502, for example, include a human-computing interface and one or more other devices that interface with the human-computing interface. The user device, or user devices, are in communication with a 3^{rd} party cloud computing service 2503, which typically includes banks of server machines. Multiple user devices 2511, which correspond to multiple users 2512, can communicate with the 3^{rd} party cloud computing service 2503. A user and their device(s) form a human edge node.

The cloud computing service 2503 is in data communication with one or more data science server machines 2504. These one or more data science server machines are in communication with internal application and databases 2505, which can reside on separate server machines, or, in another example embodiment, on the data science server machines. In an example embodiment, the data science computations executed by the data science servers and the internal applications and the internal databases are considered proprietary to given organization or company, and therefore are protected by a firewall 2506. Currently known firewall hardware and software systems, as well as future known firewall systems can be used.

The data science server machines, also called data science servers, 2504 are in communication with an artificial intelligence (AI) platform 2507. The AI platform 2507 includes one or more AI application programming interfaces (APIs) 2508 and an AI extreme data (XD) platform 2509. As will be discussed later, the AI platform runs different types of machine learning algorithms suited for different functions, and these algorithms can be utilized and accessed by the data science servers 2504 via an AI API.

The AI platform also is connected to various data sources 207, which may be 3^{rd} party data sources or internal data sources, or both. Non-limiting examples of these various data sources include: news servers, radio networks, television channel networks, video networks, IoT data, enterprise databases, social media data, etc. In an example embodiment, the AI XD platform 2509 ingests and processes the different types of data from the various data sources. An example embodiment of an infrastructure of an AI XD platform is described in U.S. patent application No. 62/472,349 filed Mar. 16, 2017 and incorporated herein by reference. Another example embodiment of an infrastructure of an AI XD platform is described in PCT Application No. PCT/US2018/022616, filed on Mar. 15, 2018 and tilted “EDGE DEVICES, SYSTEMS AND METHODS FOR PROCESSING EXTREME DATA”, and incorporated herein by reference. In an example embodiment, the human edge nodes are nodes within the AI XD platform. In other words, computations can be distributed across the human edge nodes, which each have computational resources (e.g. hardware and software resources).

In an example embodiment, the network of the servers 2503, 2504, 2505, 2506, 2507 and optionally 207 make up a data enablement system. The data enablement system provides relevant to data to the user devices, amongst other things. In an example embodiment, all of the servers 2503, 2504, 2505, 2506 and 2507 reside on cloud servers.

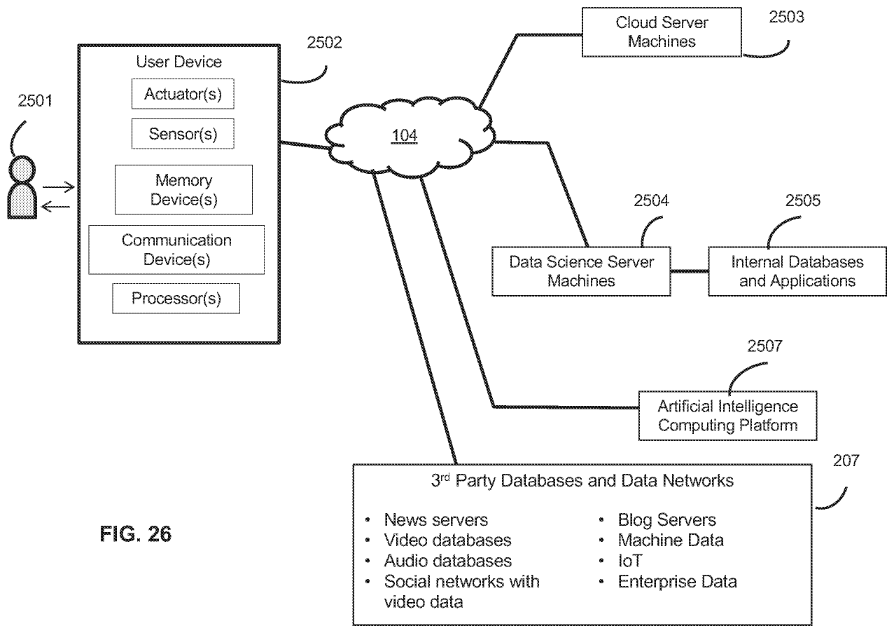

Turning to FIG. 26, another example of the servers and the devices are shown in a different data networking configuration. The user device 2502, the cloud computing servers 2503, the data science servers 2504, AI computing platform 2507, and the various data sources 2510 are able to transmit and receive data via a network 104, such as the Internet. In an example embodiment, the data science servers 2504 and the internal applications and databases 2505 are in communication with each other over a private network for enhanced data security. In another example embodiment, the servers 2504 and the internal applications and the databases 2505 are in communication with each other over the same network 104.

As shown in FIG. 26, example components of the user device 2502 include one or more processors, one or more memory devices, and one or more communication devices. Preferably, one or more of the processors can execute computations for machine learning. For example, a processor is a graphics processing unit (GPU) that performs parallel computations. In another example, a processor is a neuromorphic chip. The user device also includes, for example, sensors that depend on the type of the device (e.g. the type of human-computing interface, or the type of device controlled by the interface, or both).

The user device can also have output devices, such as actuators, electro stimulators, audio speakers, electro-mechanical devices, light projectors, display devices, motors, etc.

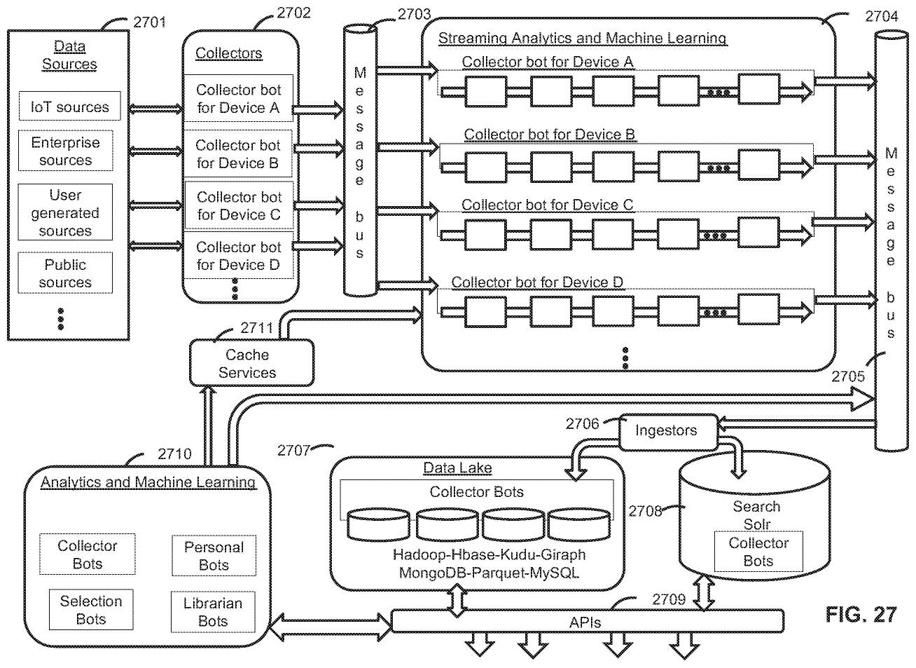

FIG. 27 shows a more detailed example computing architecture of the data enablement platform, which can be incorporated into the above computing systems.

In FIG. 27, an example computing architecture is provided for collecting data and performing machine learning on the same. This architecture, for example, is utilized in the AI platform 2507 and the data science servers 2504.

The architecture in FIG. 27 includes multiple data sources 2701. For example, data sources include those that considered part of any one or more of: the IoT data sources, the enterprise data sources, the human edge node network data sources, and the public data sources (e.g. public websites and data networks).

In particular, each one of the collector bots in the data collectors module 2702 collect data specific to a certain device. For example, one collector bot obtains data in relation Device A, and another collector both obtains data in relation to Device B.

The collector bots operate in parallel to generate parallel streams or threads of collected data. The collected data is transmitted via a message bus 2703 to a distributed streaming analytics engine 2704, which applies various data transforms and machine learning algorithms. For example, for the collector bot for Device, the streaming analytics engine 2704 has modules to transform the incoming video data, apply language detection, apply movement detection, add custom tags to the incoming data, detect trends, and extract objects and meaning from images and video. Other collector bots can have the same streaming analytics modules, or different ones. For example, another collector bot has a Surfacing analytics module, a Trend detector analytics module, Recommend analytics module, an Inference analytics module, a Predict analytics module, and an Action analytics module (collectively called STRIPA). It can be appreciated that different data sources require different reformatting protocols. Each collector bot processes its data using streaming analytics in parallel to the other search bots. This continued parallelized processing by the collector bots allows for the data enablement platform to process large amounts of data from different data sources in realtime, or near realtime.

In an example implementation, the engine 2704 is structured using one or more of the following big data computing approaches: NiFi, Spark and TensorFlow.

NiFi automates and manages the flow of data between systems. More particularly, it is a real-time integrated data logistics platform that manages the flow of data from any source to any location. NiFi is data source agnostic and supports different and distributes sources of different formats, schemas, protocols, speeds and sizes. In an example implementation, NiFi operates within a Java Virtual Machine architecture and includes a flow controller, NiFi extensions, a content repository, a flowfile repository, and a provenance repository.

Spark, also called Apache Spark, is a cluster computing framework for big data. One of the features of Spark is Spark Streaming, which performs streaming analytics. It ingests data in mini batches and performs resilient distributed dataset (RDD) transformations on these mini batches of data.

TensorFlow is software library for machine intelligence developed by Google. It uses neural networks which operate on multiple central processing units (CPUs), GPUs and tensor processing units (TPUs).

Analytics and machine learning modules 2710 are also provided to ingest larger volumes of data that have been gathered over a longer period of time (e.g. from the data lake 2707). In particular, collector bots obtain user interaction data to set parameters for filtering or processing algorithms, or to altogether select filtering or processing algorithms from an algorithms library. The collector bots, for example, use one or more of the following data science module to extract classifications from the collected data: an inference module, a sessionization module, a modeling module, a data mining module, and a deep learning module. These modules can also, for example, be implemented by NiFi, Spark or TensorFlow, or combinations thereof. In an example embodiment, unlike the modules in the streaming analytics engine 2704, the computations done by the modules 2710 are not streaming. In particular, the computations of any one or more of the collector bots, personal bots, selection bots, correction bots, and librarian bots are part the modules 2710. The results outputted by the modules 2710 are stored in memory (e.g. cache services 2711), which then transmitted to the streaming analytics engine 2704.

The results outputted by the streaming analytics engine 2704, are transmitted to ingestors 2706, via the message bus 2705. The outputted data from the analytics and machine learning modules 2710 are also transmitted to the ingestors 2706 via the message bus 2705.

The ingestors 2706 organize and store the data into the data lake 2707, which comprise massive database frameworks. Non-limiting examples of these database frameworks include Hadoop, HBase, Kudu, Giraph, MongoDB, Parquet and MySQL. The data outputted from the ingestors 2706 may also be inputted into a search platform 2708. A non-limiting example of the search platform 2708 is the Solr search platform built on Apache Lucene. The Solr search platform, for example, provides distributed indexing, load balanced querying, and automated failover and recovery.

Data from the data lake and the search engine are accessible by API services 2709.

In an example embodiment, the computing platform 105 and the human edge nodes generate immutable data. For example, the biological data and the outputted data are stored on a distributed ledger (e.g. a blockchain or other immutable data protocol), which is stored across the multiple human edge nodes.

In another example embodiment, an intelligent edge node device, like a human-computing interface or a device in communication thereto, is provided that includes: memory that stores data science algorithms and local data that is first created directly or indirectly by the intelligent edge node device; one or more processors that are configured to at least perform localized decision science using the data science algorithms to process the local data; and a communication device. The communication device communicates with other intelligent edge node devices in relation to one or more of: the data science algorithms, the processing of the local data, and an anomalous result pertaining to the local data.

In an example aspect, the one or more processors (e.g. SOCs) convert the local data to microcode and the communication device transmits the microcode to the other intelligent edge node devices. In another example aspect, the one or more processors convert the one or more data science algorithms to microcode and the communication device transmits the microcode to the other intelligent edge node devices. In another example aspect, the communication device receives microcode and the one or more processors perform local autonomous actions utilizing the microcode, wherein the microcode is at least one of new data and a new data science algorithm. In another example aspect, the memory or the one or more processors, or both, are flashable with one or more new data science algorithms. In another example aspect, the memory stores an immutable ledger that is distributed on the intelligent edge node device and the other intelligent edge node devices. In another example aspect, the local data is biological-related data that is stored on the immutable ledger.

In another example, the human edge node system 100 processes vast amounts of data (e.g. biological related data) to provide distributed and autonomous decision based actions. The system 100 includes: a plurality of intelligent human edge nodes like a human-computing interface or a device in communication thereto, wherein at least one of the plurality of intelligent edge nodes is inserted at a point where local data is first created and wherein the at least one of the plurality of intelligent human edge nodes is configured to perform localized decision science (e.g. the personal bot, the human model template computations, etc.) related to the local data. The system 100 also include a plurality of intelligent networks for transmitting data to and from the at least one of the plurality of intelligent human edge nodes, wherein at least one of the plurality of intelligent networks has embedded intelligence and wherein the transmitted data is based at least in part on the local data. The system 100 also includes a plurality of intelligent message buses interconnected with the at least one of the plurality of intelligent human edge nodes and the at least one of the intelligent networks, wherein at least one of the plurality of intelligent message buses are configured to perform autonomous actions based at least on the transmitted data.

In an example aspect, the at least one of the plurality of intelligent edge nodes is configured to create local data and to execute the localized decision science to evaluate the local data.

It is appreciated that these computing architectures are for example. Other computing architectures can also be used to accelerate the processing of data obtained by human-computing interfaces.

It will be appreciated that any module or component exemplified herein that executes instructions may include or otherwise have access to computer readable media such as storage media, computer storage media, or data storage devices (removable and/or non-removable) such as, for example, magnetic disks, optical disks, or tape. Computer storage media may include volatile and non-volatile, removable and non-removable media implemented in any method or technology for storage of information, such as computer readable instructions, data structures, program modules, or other data. Examples of computer storage media include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by an application, module, or both. Any such computer storage media may be part of the servers or devices or accessible or connectable thereto. Any application or module herein described may be implemented using computer readable/executable instructions that may be stored or otherwise held by such computer readable media.

Below are general example embodiments and related example aspects.

In a general example embodiment, a computing system is provided for provisioning control a device using biological input data. The computing system includes: a datastore comprising a library of human model templates that process biological input data to control the device; a collector bot to collect data from multiple data sources, the collected data comprising grouped instances of biological input data and output data to control the device; a librarian bot associated with the library to modify one or more of the human model templates based on the collected data; and a publisher bot to transmit the modified one or more human model templates to a given human edge node that uses the device.

In an example aspect, the multiple data sources include multiple other human edge nodes each having instances of a human-computing interface that detects biological input data and the device, which is controllable by the input data.

In another example aspect, the given human edge node comprises hardware and software that is associated with a human-computer interface device that detects the biological input data.

In another example aspect, the biological input data corresponds to brain signals. In another example aspect, the biological input data corresponds to nerve signals. In another example aspect, the biological input data corresponds to chemical signals. In another example aspect, the biological input data corresponds to eye movements. In another example aspect, the biological input data corresponds to micro-body movements. In another example aspect, the biological input data corresponds to subvocalizations. In another example aspect, the biological input data corresponds to a combination of two or more of: brain signals, nerve signals, muscle signals, chemical signals, eye movements, micro-body movements, and subvocalizations.

In another example aspect, the device is a robotic prosthetic limb. In another example aspect, the device is robot. In another example aspect, the device is a language device that outputs words via an audio speaker or a display. In another example aspect, the device is a muscle stimulating device. In another example aspect, the device is a virtual avatar of a person. In another example aspect, the device is a virtual avatar of a thing.

In another example aspect, a given human model template comprises a controller model associated with controlling the device, and the controller model comprises computation parameters that are modifiable by the librarian bot.

In another example aspect, the given human model template further comprises a pre-processor that pre-processes the biological input data and outputs pre-processed data to the controller model, and the pre-processor comprises pre-processing parameters that are modifiable by the librarian bot.

In another example aspect, the given human model template further comprises a post-processor that post-processes the controller model outputs, and the post-processor comprising post-processing parameters that are modifiable by the librarian bot.

In another example aspect, different ones of the human model templates in the library correspond to different attributes of humans.

In another example aspect, the different attributes of humans include one or more of: age, sex, dimensions, and weight.

In another example aspect, the system further includes a selection bot that selects one or more selected human model templates from the library and transmits the one or more selected human model templates to the given human edge node.

In another example aspect, the selection bot selects the one or more selected human model templates based on at least user data associated with the given human edge node.

In another example aspect, the user data comprises one or more of: age, sex, dimensions, and weight.

In another example aspect, the user data comprises health records.

In another example aspect, the user data comprises social media data.

In another general example embodiment, a human edge node system is provided that includes: memory that stores a human model template comprising computations for processing biological related inputs to control a device in communication with the human edge node system and a communication device to receive the human model template. The human edge node system also includes one or more processors that performs localized data science to execute the computations in the human model template to process incoming biological related inputs and to generate outputs that resultantly drive the device, and the one or more processors customizing the human model template based on data collected by at least one of a human-computing interface that collects the biological related inputs and the device that is controlled by the human-computing interface.

In an example aspect, the human model template is transmittable by a computing platform, and the communication device transmits the customized human model template or the data collected by the human edge node, or both, to the computing platform.

In another example aspect, the biological related inputs correspond to brain signals. In another example aspect, the biological related inputs correspond to nerve signals. In another example aspect, the biological related inputs correspond to chemical signals. In another example aspect, the biological related inputs correspond to eye movements. In another example aspect, the biological related inputs correspond to micro-body movements. In another example aspect, the biological related inputs correspond to subvocalizations. In another example aspect, the biological related inputs correspond to a combination of two or more of: brain signals, nerve signals, muscle signals, chemical signals, eye movements, micro-body movements, and subvocalizations.

In another example aspect, the human-computer interface is a brain computer interface. In another example aspect, the human-computer comprises one or more sensors placed in the brain or on the brain, or both, to detect brain signals. In another example aspect, the human-computer interface comprises one or more sensors placed exterior to the skull to detect brain signals. In another example aspect, the human-computer interface comprises one or more sensors placed a nerve or within a nerve, or both, to detect nerve signals. In another example aspect, the human-computer interface comprises one or more sensors placed on skin to detect nerve signals. In another example aspect, the human-computer interface comprises one or more sensors placed within a muscle or on a muscle, or both, to detect muscle signals. In another example aspect, the human-computer interface comprises one or more sensors placed on skin to detect muscle signals.

In another example aspect, human edge node system includes the human-computer interface that obtains the biological related inputs.

In another example aspect, the device is a robotic prosthetic limb. In another example aspect, the device is robot. In another example aspect, the device is a language device that outputs words via an audio speaker or a display. In another example aspect, the device is a muscle stimulating device. In another example aspect, the device is a virtual avatar of a person. In another example aspect, the device is a virtual avatar of a thing.

In another example aspect, the device is part of the human edge node system.

In another example aspect, the device comprises one or more sensors for sensing context data.

In another example aspect, the context data comprises audio data. In another example aspect, the context data comprises image data. In another example aspect, the context data comprises temperature data. In another example aspect, the context data comprises tactile data. In another example aspect, the context data comprises RADAR data. In another example aspect, the context data comprises LiDAR data.

In another example aspect, the one or more processors customize the human model template based on the context data collected by the device.

In another example aspect, multiple human model templates are stored in the memory, and the one or more processors use the context data to select a context-relevant human model template from the multiple human model templates, and the context-relevant human model template is used to drive the device.

In another general example embodiment, a human edge node system is provided that includes: memory that stores a human model template comprising computations for processing data inputs to control a human-computer interface device; a communication device to receive the human model template; and one or more processors that locally executes the computations in the human model template to generate outputs that resultantly drive the human-computer interface device, and the one or more processors customizing the human model template based on biological related data detected in response to driving the human-computer interface device. The driving of the human-computer interface device affects a human's biological system.

In an example aspect, the human-computer interface device is part of the human edge node.

In another example aspect, the human-computer interface device is a brain stimulation device. In another example aspect, the human-computer interface device is a muscle stimulation device. In another example aspect, the human-computer interface device is a nerve stimulation device. In another example aspect, the human-computer interface device is a chemical stimulation device. In another example aspect, the human-computer interface device releases one or more drugs. In another example aspect, the human-computer interface device stimulates at least one of new neurons, new synapses, and new axions to stimulate new neural circuits.

In another general example embodiment, a computing system is provided for provisioning control of multiple devices using biological input data from a human edge node. The computing system comprises: a datastore comprising a library of human model templates that process biological input data to control the multiple devices; a collector bot to collect data from multiple data sources, the collected data comprising grouped instances of biological input data and output data to control the multiple devices; a librarian bot associated with the library to modify one or more of the human model templates based on the collected data; and a publisher bot to transmit the modified one or more human model templates to a given human edge node that controls the multiple devices.

In an example aspect, the multiple devices are of a same type of device.

In another example aspect, the multiple devices comprise different types of devices.

In other words, a human edge node is able to control multiple devices. For example, a user can use a human-computer interface to control a swarm of devices (e.g. robots, drones, etc.) at the same time. The devices can be of the same type or can be of a different type.

It will be appreciated that different features of the example embodiments of the system and methods, as described herein, may be combined with each other in different ways. In other words, different devices, modules, operations, functionality and components may be used together according to other example embodiments, although not specifically stated.

The steps or operations in the flow diagrams described herein are just for example. There may be many variations to these steps or operations according to the principles described herein. For instance, the steps may be performed in a differing order, or steps may be added, deleted, or modified.

It will also be appreciated that the examples and corresponding system diagrams used herein are for illustrative purposes only. Different configurations and terminology can be used without departing from the principles expressed herein. For instance, components and modules can be added, deleted, modified, or arranged with differing connections without departing from these principles.

Although the above has been described with reference to certain specific embodiments, various modifications thereof will be apparent to those skilled in the art without departing from the scope of the claims appended hereto.

1. A computing system for provisioning control of a device using biological input data, comprising:
a datastore comprising a library of human model templates that process biological input data to control the device;
a collector bot to collect data from multiple data sources, the collected data comprising grouped instances of biological input data and output data to control the device;
a librarian bot associated with the library to modify one or more of the human model templates based on the collected data; and
a publisher bot to transmit the modified one or more human model templates to a given human edge node that uses the device.

2. The computing system of claim 1 wherein the multiple data sources include multiple other human edge nodes each having instances of a human-computing interface that detects biological input data and the device, which is controllable by the input data.

3. The computing system of claim 1 wherein the given human edge node comprises hardware and software that is associated with a human-computer interface device that detects the biological input data.

4. The computing system of claim 1 wherein the biological input data corresponds to brain signals.

5. The computing system of claim 1 wherein the biological input data corresponds to nerve signals.

6. The computing system of claim 1 wherein the biological input data corresponds to chemical signals.

7. The computing system of claim 1 wherein the biological input data corresponds to eye movements.

8. The computing system of claim 1 wherein the biological input data corresponds to micro-body movements.

9. The computing system of claim 1 wherein the biological input data corresponds to subvocalizations.

10. The computing system of claim 1 wherein the biological input data corresponds to a combination of two or more of: brain signals, nerve signals, muscle signals, chemical signals, eye movements, micro-body movements, and subvocalizations.

11. The computing system of claim 1 wherein the device is a robotic prosthetic limb.

12. The computing system of claim 1 wherein the device is a robot.

13. The computing system of claim 1 wherein the device is a language device that outputs words via an audio speaker or a display.

14. The computing system of claim 1 wherein the device is a muscle stimulating device.

15. The computing system of claim 1 wherein the device is a virtual avatar of a person or a thing.

16. The computing system claim 1 wherein the device is a graphical user interface control element.

17. The computing system of claim 1 wherein a given human model template comprises a controller model associated with controlling the device, and the controller model comprises computation parameters that are modifiable by the librarian bot.

18. The computing system of claim 17 wherein the given human model template further comprises a pre-processor that pre-processes the biological input data and outputs pre-processed data to the controller model, and the pre-processor comprises pre-processing parameters that are modifiable by the librarian bot.

19. The computing system of claim 17 wherein the given human model template further comprises a post-processor that post-processes the controller model outputs, and the post-processor comprising post-processing parameters that are modifiable by the librarian bot.

20. The computing system of claim 1 wherein different ones of the human model templates in the library correspond to different attributes of humans.

21. (canceled)

22. The computing system of claim 1 further comprising a selection bot that selects one or more selected human model templates from the library and transmits the one or more selected human model templates to the given human edge node.

23. The computing system of claim 22 wherein the selection bot selects the one or more selected human model templates based on at least user data associated with the given human edge node.

24. (canceled)

25. (canceled)

26. The computing system of claim 23 wherein the user data comprises social media data.

27.-70. (canceled)
